(19) United States
(12) Patent Application Publication
(10) Pub. No.: US 2010/0234947 A1
(43) Pub. Date: Sep. 16, 2010

(54) IMPLANTED MEDICAL DEVICE ESPECIALLY USED IN COSMETIC SURGERY

(76) Inventors: Yaniv Ben Rubi, Shimshit (IL); Li-Hai Kats, Haifa (IL); Roni Shabat, Kfar Yeheskel (IL)

Correspondence Address: The Law Office of Michael E. Kondoudis, 888 16th Street, N.W., Suite 800, Washington, DC, 20006, US

(21) Appl. No.: 12/670,009

(22) PCT Filed: Jul. 27, 2008
(86) PCT No.: PCT/IL08/01038

§ 371 Date: Jun. 2, 2010

Related U.S. Application Data
(60) Provisional application No. 60/951,960, filed on Jul. 26, 2007.

(51) Int. Cl. A61F 2/02

(52) U.S. Cl. 623/11.11

(57) ABSTRACT

The present invention provides an implantable medical device adapted for use in surgical operations, said device being implantable in a predetermined body portion to be treated; said predetermined body portion is characterized by a first extremity and by a second extremity, wherein said device comprises (a) at least one element having a body, a distal end, and a proximal end; said body being characterized by (i) at least one inactivated position; and, (ii) a plurality of activated positions; (b) at least one anchoring means coupled to said distal end of said element, adapted to anchor said distal end to said first extremity in said predetermined body portion; and, (c) at least one anchoring means coupled to said proximal end of said element, adapted to anchor said proximal end to said second extremity in said predetermined body portion; said element is at least partially reconfigurable from said activated position to said inactivated position and/or from said plurality of inactivated positions to said plurality of activated positions such that the distance between said first extremity and said second extremity is alterable.

### FIELD OF THE INVENTION

The present invention relates in general to the field of minimally invasive plastic and or cosmetic surgery. More specifically the present invention relates to methods for manipulating tissue placement and affixing tissue by springs or stent-like devices and to conducting tools for delivering such springs through the tissue and fixing them within designated zones, for purposes of smoothing wrinkles, reconstructive purposes, and the like.

### BACKGROUND OF THE INVENTION

Humankind has long devised arts to increase physical appeal. Makeup for instance has been in existence for at least 4,000 years. Moisturizers, skin toners, and a seemingly endless series of salves, creams, lotions, potions, injections, mud-packs, fruit treatments, and others promise to restore the tone and vigour of youthful skin. More recently, reconstructive surgery for cosmetic purposes has found widespread acceptance. Various methods have been devised in this direction, often involving the subcutaneous stretching of skin. Employing anchored and barbed polypropylene sutures, known as contour threads™ for removing laxity of a face and or neck, is a known surgical procedure, but holds several drawbacks. Therefore any method and or device providing for such surgical operations and their improvement shall be beneficial.

For example, US patent application US20070293892 discloses a surgical thread for plastic surgery which effectively removes sagging and wrinkling of skin. The device comprises a thread shaped member comprised of a thread body, to be implanted in the inside layers of the skin, partially formed in its longitudinal direction with projections for anchoring in the inside skin layers, wherein at least the thread body is comprised of an absorbable thread, and the thread body or the projections are formed with residual film parts which will not be absorbed by the inside skin layers, and a method of imparting tension to the skin using the same. In FIG. 1 this device is depicted.

However the system is somewhat primitive in its capabilities. For example, it cannot provide independent control over tension between successive anchoring projections. Nor does it allow for both tension and compression to be provided by the same device. Finally the anchoring projections, being simple hooks, do not allow for certain operations such as gathering tissue together by a single anchor.

US patent application 2007/067045 discloses an implant that reduces wrinkles, in the shape of a cylinder with a constant or varying cross-section and length. The implant contains a gel of limited flow capability. The implant can also be a balloon that may or may not have multiple compartments optionally filled with fluid. However the system cannot provide independent control, over tension between given points. Nor does it allow for both tension and compression to be provided by the same device. Finally the device does not allow for certain operations such as gathering tissue together.

PCT application WO06065837 provides a cosmetic implant comprising a filament made from a biocompatible elastomer. The elastomeric filament can be injected or pulled under one or more wrinkles. Once implanted under the wrinkle(s), the filament lifts and supports the tissue above it. Such lifting lessens (and possibly removes altogether) the appearance of the wrinkle(s). Again however the system cannot provide independent control over tension between given anchoring points. Nor does it allow for both tension and compression to be provided by the same device. Finally the system does not allow for certain operations such as gathering tissue together at a point.

Therefore, there is still a long felt need for an implantable medical device adapted for remoulding and/or reconstructing both soft and/or hard tissues with controllable tensioning means and multiple independent tensioning means.

### SUMMARY OF THE INVENTION

It is one object of the present invention to provide an implantable medical device adapted for use in surgical operations, said device being implantable in a predetermined body portion to be treated; said predetermined body portion is characterized by a first extremity and by a second extremity, wherein said device comprises (a) at least one element having a body, a distal end, and a proximal end; said body being characterized by (i) at least one inactivated position; and, (ii) a plurality of activated positions; (b) at least one anchoring means coupled to said distal end of said element, adapted to anchor said distal end to said first extremity in said predetermined body portion; and, (c) at least one anchoring means coupled to said proximal end of said element, adapted to anchor said proximal end to said second extremity in said predetermined body portion; said element is at least partially reconfigurable from said activated position to said inactivated position and/or from said plurality of inactivated positions to said plurality of activated positions such that the distance between said first extremity and said second extremity is alterable.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is another object of the present invention to provide the implantable medical device as defined above, useful in medical procedure for repair of apertures in hard biological tissue selected from a group consisting of orthopaedic surgeries, dental surgeries, soft and hard tissues reattachments.

It is another object of the present invention to provide the implantable medical device as defined above, wherein said at least two anchoring means are positioned at an angle A relatively to each other.

It is another object of the present invention to provide the implantable medical device as defined above, wherein said angle A is greater than about 0 degrees and lower than about 180 degrees, especially at an angle 180 degrees.

It is another object of the present invention to provide the implantable medical device as defined above, wherein said element is selected from a group consisting of a spring, an inflatable element, fillable element.

It is another object of the present invention to provide the implantable medical device as defined above, wherein said element additionally comprises sealing means.

It is another object of the present, invention to provide the implantable medical device as defined above, wherein said element may be transformed by means selected from group consisting of: stretching, compression, inflation, deflation.

It is another object of the present invention to provide the implantable medical device as defined above, wherein said element is made of material selected from a group consisting of shape memory materials, biodegradable materials, biocompatible materials or any combination thereof.

It is another object of the present invention to provide the implantable medical device as defined above, additionally comprising at least one anchoring means coupled to said body of at least one of said elements.

It is another object of the present invention to provide the implantable medical device as defined above, wherein said anchoring means are mechanically linked.

It is another object of the present invention to provide an implantable medical device adapted for use in surgical operations; said device being implantable in a predetermined body portion to be treated; wherein said device comprises (a) at least one stationary anchoring means; (b) at least one movable anchoring means in mechanical communication with said stationary anchoring means via at least one shaft; said movable anchoring means is adapted to reciprocally move along the longitudinal axis of said shaft; and, (c) at least one element having a body, a distal end and a proximal end; said body is characterized by at least one inactivated position and a plurality of activated positions; said element is coupled to said movable anchoring means at least one end of said element; said element is at least partially reconfigurable from said activated position to said inactivated position and/or from said plurality of inactivated positions to said plurality of activated positions.

It is another object of the present invention to provide the implantable medical device as defined above, additionally comprising means for activating said element, such that said body is reconfigured from said activated position to said inactivated position and/or from said inactivated position to said activated position

It is another object of the present invention to provide the implantable medical device as defined above, wherein said means for activating is selected from a group consisting of inflating or deflating means, filling or withdrawing means, pressure, tension, force, heat, luminescence, change of Ph, application of magnetic field, application of electric field, voltage.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is another object of the present invention to provide the implantable medical device as defined above, useful in medical procedure for repair aperture in hard biological tissue selected from a group consisting of orthopaedic surgeries, dental surgeries, soft and hard tissues reattachments.

It is another object of the present invention to provide the implantable medical device as defined above, wherein said at least two anchoring means are positioned at an angle A relatively to each other.

It is another object of the present invention to provide the implantable medical device as defined above, wherein said angle A is greater than about 0 degrees and lower than about 180 degrees, especially at an angle of 0 degrees.

It is another object of the present invention to provide the implantable medical device as defined above, wherein said element is selected from a group consisting a spring, an inflatable element, fillable element.

It is another object of the present invention to provide the implantable medical device as defined above, wherein said element additionally comprises sealing means.

It is another object of the present invention to provide the implantable medical device as defined above, wherein said element is made of material selected from a group consisting of shape memory materials, biodegradable materials, biocompatible materials or any combination thereof.

It is another object of the present invention to provide an implantable medical device adapted for use in surgical operations; said device being implantable in a predetermined body portion to be treated, said predetermined body portion is characterized by a first extremity and by a second extremity; wherein said device comprises (a) at least one shaft having a proximal end and a distal end; (b) at least one stationary anchoring means coupled to said shaft in said proximal end; said stationary anchoring means is adapted to be at least partially reversibly anchored to said first extremity in said predetermined body portion; and, (c) at least one movable anchoring means coupled to said distal end of said shaft; said movable anchoring means is adapted to be at least partially reversibly anchored to said second extremity in said predetermined body portion, said movable anchoring means are adapted to reciprocally move along the longitudinal axis of said shaft such that the distance between said first extremity and said second extremity is alterable.

It is another object of the present invention to provide the implantable medical device as defined above, additionally comprising means coupled to said movable anchoring means and enables said movement of said movable anchoring means along the longitudinal axis of said shaft.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is another object of the present invention to provide the implantable medical device as defined above, useful in medical procedure for repair aperture in hard biological tissue selected from a group consisting of orthopaedic surgeries, dental surgeries, soft and hard tissues reattachments.

It is another object of the present invention to provide the implantable medical device as defined above, wherein said at least two anchoring means are positioned at an angle A relatively to each other.

It is another object of the present invention to provide the implantable medical device as defined above, wherein said angle A is greater than about 0 degrees and lower than about 180 degrees, especially at an angle of 0 degrees.

It is another object of the present invention to provide an implantable medical device adapted for use in surgical operations; said device being implantable in a predetermined body portion to be treated, said predetermined body portion is characterized by a first extremity and by a second extremity; wherein said device comprises (a) at least one shaft having a proximal end and a distal end; said shaft is provided with ratchets located on said distal end of said shaft; (b) at least one stationary anchoring means coupled to said shaft in said proximal end, said stationary anchoring means is adapted to be at least partially reversibly anchored to said first extremity in said predetermined body portion; and, (c) at least one movable anchoring means coupled to said distal end of said shaft; said movable anchoring means is adapted to be at least partially reversibly anchored to said second extremity in said predetermined body portion; said movable anchoring means are adapted to unidirectionally move within said shaft such that the distance between said first extremity and said second extremity is alterable.

It is another object of the present invention to provide the implantable medical device as defined above, additionally comprising means coupled to said movable anchoring means and enables said movement of said movable anchoring means along the longitudinal axis of said shaft.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is another object of the present invention to provide the implantable medical device as defined above, useful in medical procedure for repair aperture in hard biological tissue selected from a group consisting of orthopaedic surgeries, dental surgeries, soft and hard tissues reattachments.

It is another object of the present invention to provide the implantable medical device as defined above, wherein said at least two anchoring means are positioned at an angle A relatively to each other.

It is another object of the present invention to provide the implantable medical device as defined above, wherein said angle A is greater than about 0 degrees and lower than about 180 degrees, especially at an angle of 0 degrees.

It is another object of the present invention to a method for treating tissues in a predetermined body portion during surgical operations, said predetermined body portion to be treated is characterized by a first extremity and a second extremity. The method comprising steps selected inter alia from (a) providing an implantable medical device comprising (i) at least one element having a body, a distal end, and a proximal end; said body is characterized by (i) at least one inactivated position; and, (ii) a plurality of activated positions; (ii) at least one anchoring means coupled to said distal end of said element; and, (iii) at least one anchoring means coupled to said proximal end of said element; (b) introducing said implantable medical device in said activated position or in said inactivated position to said body portion to be treated; (c) at least partially reversibly anchoring said distal end of said implantable medical device to said first extremity in said predetermined body portion via one of said anchoring means; (d) reconfiguring said body from said activated position to said inactivated position and/or from said inactivated position to said activated position; and, (e) at least partially reversibly anchoring said proximal end of said implantable medical device to said second extremity in said predetermined body portion via one of said anchoring means, thereby imparting tension and altering the distance between said first extremity and said second extremity thus, treating said tissue of said body portion.

It is another object of the present invention to provide the method as defined above, additionally comprising step of releasing either one of said anchoring means.

It is another object of the present invention to provide the method as defined above, additionally comprising the step of reconfiguring said at least one element from said activated position to said inactivated position and/or from said inactivated position to said activated position.

It is another object of the present invention to provide a method for treating tissues in a predetermined body portion during surgical operations. The method comprising steps selected inter alia from (a) providing an implantable medical device comprising (i) at least one element having a body, a distal end, and a proximal end; said body is characterized by (i) at least one inactivated position; and, (ii) a plurality of activated positions; (ii) at least one anchoring means coupled to said distal end of said element; and, (iii) at least one anchoring means coupled to said proximal end of said element; (b) introducing said implantable medical device in said activated position or in said inactivated position to said body portion to be treated; (c) at least partially reversibly anchoring said distal end of said implantable medical device to said first extremity in said predetermined body portion via one of said anchoring means; (d) at least partially reversibly anchoring said proximal end of said implantable medical device to said second extremity in said predetermined body portion via one of said anchoring means; (e), reconfiguring said body from said activated position to said inactivated position and/or from said inactivated position to said activated position; imparting tension and altering the distance between said first extremity and said second extremity thereby treating said tissue of said body portion.

It is another object of the present invention to provide the method as defined above, additionally comprising step of releasing either one of said anchoring means.

It is another object of the present invention to provide method as defined above, additionally comprising the step of reconfiguring said at least one element from said activated position to said inactivated position and/or from said inactivated position to said activated position.

It is another object of the present invention to provide a method for treating tissues in a predetermined body portion during surgical operations, said predetermined body portion to be treated is characterized by a first extremity and a second extremity. The method comprising steps selected inter alia from (a) providing an implantable medical device having (i) at least one stationary anchoring means; (ii) at least one movable anchoring means in mechanical communication with said stationary anchoring means via at least one shaft; and, (iii) at least one element having a body, a distal end and a proximal end, said body is characterized by at least one activated position and at least one inactivated position; (b) coupling said movable anchoring means to said element at said distal end or said proximal end; (c) inserting said implantable medical device in said activated position or in said inactivated position into said body portion to be treated; (d) at least partially reversibly anchoring said stationary anchoring means to said first extremity in said body portion to be treated; (e) at least partially reversibly anchoring said movable anchoring means to said second extremity in said body portion to be treated; and, (f) reversibly activating said body of said at least one element from said activated position to said inactivated position and/or from said inactivated position to said activated position such that said movable anchoring means is reciprocally moving along said shaft; thereby imparting tension and altering the distance between said first extremity and said second extremity thus, treating said tissue of said body portion.

It is another object of the present invention to provide method as defined above, additionally comprising step of releasing either one of said anchoring means.

It is another object of the present invention to provide method as defined above, additionally comprising the step of reconfiguring said at least one element from said activated position to said inactivated position and/or from said inactivated position to said activated position.

It is another object of the present invention to provide a method for treating tissues in a predetermined body portion during surgical operations, said predetermined body portion to be treated is characterized by a first extremity and a second extremity. The method comprising steps selected inter alia from (a) providing an implantable medical device having (i) at least one stationary anchoring means; (ii) at least one movable anchoring means in mechanical communication with said stationary anchoring means via at least one shaft; (b) inserting said implantable medical device into said body portion to be treated; (c) at least partially reversibly anchoring said stationary anchoring means to said first extremity in said body portion to be treated; (d) at least partially reversibly anchoring said movable anchoring means to said second extremity in said body portion to be treated; and, (e) reciprocally moving said movable anchoring means along said shaft; thereby imparting tension and altering the distance between said first extremity and said second extremity thus, treating said tissue of said body portion.

It is another object of the present invention to provide the method as defined above, additionally comprising step of releasing either one of said anchoring means.

It is another object of the present invention to provide the method as defined above, additionally comprising the step of reconfiguring said at least one element from said activated position to said inactivated position and/or from said inactivated position to said activated position.

It is another object of the present invention to provide an implantable medical device adapted for use in surgical operations; said device being implantable in a predetermined body portion to be treated; wherein said device comprises (a) a hollow element having a biocompatible outer surface, said hollow element is characterised by an inactivated position and a plurality of activated positions; (b) a plurality of protrusions coupled to said outer surface, adapted to anchor said hollow element to said body portion to be treated; (c) coupling means accommodated within said hollow element, for imparting mechanical tension on said element.

It is another object of the present invention to provide the implantable medical device as defined above, wherein the shape of said element is selected from a group' consisting of: zigzag shaped, square shape, C shape, of regular or irregular spiral, lumen, pipe, crescent, linear, bagel-like (annular), or star-like cross section, planar or proximally planar shaped barriers of any size, shape or type, 3D configurations, and multi-dimensional configurations comprising a plurality of regular or irregular two or more 2D planes and/or 3D spatial members interlinked together to form a continuous member.

It is another object of the present invention to provide the implantable medical device as defined above, wherein said element is selected from a group consisting a spring, an inflatable element, fillable element.

It is another object of the present invention to provide the implantable medical device as defined above, wherein said element additionally comprises sealing means.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is another object of the present invention to provide the implantable medical device as defined above, useful in medical procedure for repair aperture in hard biological tissue selected from a group consisting of orthopaedic surgeries, dental surgeries, soft and hard tissues reattachments.

It is another object of the present invention to provide the implantable medical device as defined above, wherein said elements are made of material selected from a group consisting of shape memory materials, biodegradable materials, biocompatible materials or any combination thereof.

It is another object of the present invention to provide a method for treating tissues in a predetermined body portion during surgical operations. The method comprising steps selected inter alia from (a) providing at least one implantable medical device comprises (i) a hollow element having a biocompatible outer surface, said hollow element is characterised by an inactivated position and a plurality of activated positions; (ii) a plurality of protrusions coupled to said outer surface, adapted to anchor said hollow element to said body portion to be treated; and, (iii) coupling means accommodated within said hollow element, for imparting mechanical tension on said element; (b) threading said coupling means through said hollow element; (c) incorporating said threaded implantable medical device in said inactivated position within an introducer; (d) introducing said implantable medical device via said introducer into said body portion to be treated; (e) at least partially anchoring said implantable medical device to said tissue in said body portion to be treated via said plurality of protrusions; (f) imparting mechanical tension onto said tissue via said coupling means, thereby treating said tissue in said predetermined body portion.

It is another object of the present invention to provide a method for treating tissues in a predetermined body portion during surgical operations. The method comprising steps selected inter alia from (a) providing at least two implantable medical device, each of which comprises (i) a hollow element having a biocompatible outer surface, said hollow element is characterised by an inactivated position and a plurality of activated positions; (ii) a plurality of protrusions coupled to said outer surface, adapted to anchor said hollow element to said body portion to be treated; and, (iii) coupling means accommodated within said hollow element, for imparting mechanical tension on said element; (b) threading said coupling means through said hollow element thereby mechanically coupling pairs of said implantable medical device together; (c) incorporating said threaded implantable medical devices in said inactivated position within an introducer; (d) introducing at least one of said implantable medical device via said introducer into said body portion to be treated; (e) at least partially anchoring said implantable medical device to said tissue in said body portion to be treated via said plurality of protrusions; (f) introducing the second element via said introducer into said body portion to be treated; (g) at least partially anchoring said second element to said tissue in said body portion to be treated via said plurality of cogs; (h) altering the tension in said coupling means such that the distance between said pairs is altered and mechanical tension is imparted on said tissue, thereby treating said tissue in said predetermined body portion.

It is another object of the present invention to provide the method as defined above, additionally comprising step of repeating said steps of incorporating, introducing, anchoring and altering.

It is another object of the present invention to provide the method as defined above, wherein said step of tensing said tissue is performed by altering in an arbitrary geometric manner said pairs by means of independently fixed tension between every connected pair of said elements.

It is another object of the present invention to provide the method as defined above, additionally comprising step of affixing each of said flexible coupling means to said tissues in said predetermined body portion.

It is another object of the present invention to provide a device for treating tissues in a predetermined body portion, said device comprising (a) a magazine accommodating a plurality of implantable medical devices threaded together via coupling means; (b) means for individually introducing each one of said implantable medical devices to said predetermined body portion; (c) means for imparting mechanical tension in said coupling means between each pair of implantable medical device by stretching or relaxing said coupling means.

It is another object of the present invention to provide the device as defined above, wherein said device additionally comprises means for anchoring each of said coupling means to said elements in said predetermined body portion such that the tension between each pair of said implantable medical devices is fixed

It is another object of the present invention to provide the device as defined above wherein said means for anchoring is selected from a group consisting of: affixing remotely, affixing by hand, tying, connecting, attaching, gluing, stapling, and sewing.

It is another object of the present invention to provide the device as defined above wherein said means for individually introducing comprises (a) a shaft adapted to linearly move said implantable medical devices; and, (b) a stopper adapted to introduce a single implantable medical device to said body portion to be treated.

It is another object of the present invention to provide a fastener for use in surgical operations; said fastener being implantable in a predetermined body portion to be treated, wherein said fastener comprises (a) at least two arm positioned at an angle A relatively to one another; (b) a rigid stopper adapted to fixate said at least two arms at said angle A; (c) a plurality of protrusions coupled to at least a portion of said arms, adapted to anchor said fastener to said tissue in said body portion to be treated.

It is another object of the present invention to provide the fastener as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the fastener as defined above, useful in cosmetic medical procedure selected from a group consisting of wrinkles removal, face lifting.

It is another object of the present invention to provide the fastener as defined above, useful in medical procedure selected from a group consisting of intensive and/or immediate care for repairing apertures.

It is another object of the present invention to provide the fastener as defined above, useful in medical procedure for repair of apertures in hard biological tissue selected from a group consisting of orthopaedic surgeries, dental surgeries, soft and hard tissues reattachments.

It is another object of the present invention to provide the fastener as defined above, wherein said angle A is greater than about 0 and lower than about 180 degrees.

It is another object of the present invention to provide the an implantable medical device adapted for use in surgical operations; said device being implantable in a predetermined body portion to be treated; wherein said device comprises (a) a plurality of hollow elements having a biocompatible outer surface, each of said hollow elements is characterised by an inactivated position and a plurality of activated positions; (b) a plurality of protrusions coupled to said outer surface of each of said hollow elements, adapted to anchor each of said hollow elements said hollow elements to said body portion to be treated; (c) coupling means accommodated within each of said hollow elements, for (i) mechanically coupling pairs of said hollow elements together; and, (ii) imparting mechanical tension on each of said hollow elements.

It is another object of the present invention to provide the implantable medical device as defined above, wherein the shape of said element is selected from a group consisting of: zigzag shaped, square shape, C shape, of regular or irregular spiral, lumen, pipe, crescent, linear, bagel-like (annular), or star-like cross section, planar or proximally planar shaped barriers of any size, shape or type, 3D configurations, and multi-dimensional configurations comprising a plurality of regular or irregular two or more 2D planes and/or 3D spatial members interlinked together to form a continuous member.

It is another object of the present invention to provide the implantable medical device as defined above, wherein said element is selected from a group consisting a spring, an inflatable element, fellable element.

It is another object of the present invention to provide the implantable medical device as defined above, wherein said element additionally comprises sealing means.

It is another object of the present invention to provide the implantable medical device as defined above, wherein said surgical operations are selected from a group consisting of cosmetic surgeries, especially for remoulding and/or reconstructing both soft and/or hard tissues, repairing apertures in soft biological tissue or any combination thereof.

It is another object of the present invention to provide the implantable medical device as defined above, useful in cosmetic medical procedure selected from a group consisting of wrinkles removal, face lifting.

It is another object of the present invention to provide the implantable medical device as defined above, useful in medical procedure selected from a group consisting of intensive and/or immediate care for repairing apertures.

It is still an object of the present invention to provide the implantable medical device as defined above, useful in medical procedure for repair aperture in hard biological tissue selected from a group consisting of orthopaedic surgeries, dental surgeries, soft and hard tissues reattachments.

It is lastly an object of the present invention to provide the implantable medical device as defined above, wherein said elements are made of material selected from a group consisting of shape memory materials, biodegradable materials, biocompatible materials or any combination thereof.

### BRIEF DESCRIPTION OF THE DRAWINGS

The present invention will be understood and appreciated more fully from the following detailed description taken in conjunction with the appended drawings in which:

FIG. 1 shows prior art concerning contour threads™.

FIG. 2a-c is a side-view of a conducting tool of the present invention;

FIG. 3a-d is an isometric view of the distal end of the conducting tool;

FIG. 4 is an example of a loaded magazine;

is a side-view of an exemplary spring;

is a side-view of yet another exemplary spring;

FIG. 5a,b are illustrations of the introducer and the process of introducing anchoring elements

FIG. 6a,b are further illustrations of the introducer and the process of introducing anchoring elements

FIG. 7 is an isometric view of a conducting tool inserted under patient's skin;

FIGS. 8a-8f are isometric views of exemplary anchoring elements including sutures;

FIGS. 9a-9e are isometric views of another exemplary anchoring element in rest state;

FIGS. 10a-10d are views of coupled anchoring elements FIGS. 12a-11e show an alternative embodiment of the anchoring devices and springs

FIG. 12 presents a further embodiment of anchor mechanisms with variable degree of tension.

FIGS. 13a and 13b presents a further embodiment of anchor mechanisms with variable degree of tension provided by variable numbers of connecting elements.

FIG. 14 presents a further embodiment of an anchoring and tensioning using inflatable pistons.

FIG. 15a-c presents a further embodiment of an anchoring and tensioning mechanism using a collapsible member and allowing for a tilted configuration.

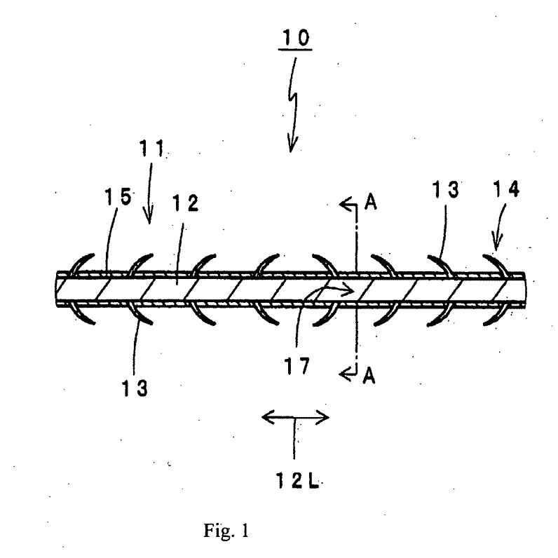

In FIGS. 16a-16c an embodiment is shown wherein anchors are forced together or apart by rotation of a rigid rod provided with a screw.

In FIGS. 17a-17d an embodiment using a sliding member to fix the angle between two arms of the anchoring mechanism is show.

In FIG. 18 an embodiment is shown wherein several anchoring elements are all pulled from a single point by coupling means.

In FIG. 19a,b an embodiment is shown wherein a bone fracture is set by means of anchoring elements of the present invention.

In FIG. 20a,b an embodiment is shown wherein a cut in the skin of the leg is closed by means of: anchoring elements of the present invention.

In FIG. 21a,b an embodiment is shown wherein a cut in the skin of the leg is closed by means of anchoring elements of another embodiment of the present invention.

While the invention is susceptible to various modifications and alternative forms, specific embodiments thereof have been shown by way of example in the drawings. The components in the drawings are not necessarily to scale, emphasis instead being placed upon clearly illustrating the principles of the present invention. It should be understood, however, that the description herein of specific embodiments is not intended to limit the invention to the particular forms disclosed, but on the contrary, the intention is to cover all modifications, equivalents, and alternatives falling within the spirit and scope of the invention.

### DETAILED DESCRIPTION OF THE PRESENT INVENTION

The following description is provided in order to enable any person skilled in the art to make use of said invention and sets forth the best modes contemplated by the inventor of carrying out this invention. Various modifications, however, will be apparent to those skilled in the art, since the generic principles of the present invention have been defined specifically to provide an implantable medical device and a method of using the same, and therefore the invention is not limited by that which is illustrated in the figures and described in the specification, but only as indicated in the accompanying claims, with the proper scope determined only by the broadest interpretation of said claims.

In accordance with the present invention a method and system for tissues and skin manipulation is provided. The method according to the invention provides for implementation of springs for smoothing skin by stretching or extending the skin and or associated tissues to reduce wrinkles and achieve aesthetic results. The system of the invention includes a conducting tool and spring magazine incorporated into the conducting tool. The spring magazine holds one or more springs to be implanted in tissues of a patient. The springs, optionally threaded with sutures, may be loaded into the spring magazine of the invention by means of a dedicating spring loader. The spring or springs are held in a nonequilibrium state, typically stretched or compressed, while being loaded in the spring magazine. The spring magazine is mounted onto a conducting tool, whereby the springs can be placed or embedded within the target tissue. On release of the springs from the magazine, they are returned to their rest position, inducing the desired aesthetic effect. A multitude of alternative embodiments of various tensioning means are furthermore provided.

The present invention provides an approach and a method in the category of minimally invasive surgical procedures. It employs a stent-like spring having spikes or cogs extending from its surface, which, is introduced subcutaneously by means of a conducting tool of the invention. When released under the skin the spring extends or retracts (depends on a particular embodiment of the spring) thereby stretching or contracting the adjacent tissue and skin. Alternately the spring remains fixed under the skin when released by the conducting tool, while the operator stretches the skin and presses downward on the skin to allow the cogs to penetrate surrounding tissues. The spring, once in place, acts as an anchor point in the tissue. The spring can hold the skin in a stretched position to prevent it from re-contracting (thus preventing wrinkles from reforming). Further embodiments allow for arbitrarily complex ‘webs’ to be created by providing a suture in each anchor point. The suture or thread connecting each spring and the successive spring can be pulled by the positioning device to reach a desired level of tension. Then the suture is affixed to the spring, establishing a fixed tension that will not change. The subsequent spring has its own section of suture and its own tension. In this way the tension between any two successive springs can be independently set. By this method an arbitrarily complex geometric remolding of subcutaneous tissue can be accomplished.

The term “inflatable element” refers hereinafter to any flexible object with the property that it can be inflated, with subsequent expansion of said object. The inflatable element can be made from materials such as rubber, latex, silicone rubber, polyurethane, chloroprene or a nylon fabric or any thermoelastomeric materials.

The term “fillable element” refers hereinafter to any flexible object with the property that it can be filled, with subsequent expansion in some dimension of said object. In contrast to an inflatable element, the wall thickness of a fillable element will generally not be affected by the process of expansion. The finable element can be made from materials such as metal, plastic, corrugated materials, and the like in the form of pistons, syringes, and the like.

The term “biocompatible materials” refers hereinafter to materials that have the ability to perform with an appropriate host response in a specific application. Biocompatible materials have the quality of not having toxic or injurious effects on biological systems.

The term “biodegradable materials” refers hereinafter to materials that are degraded by the body's enzymatic pathways through a reaction against “foreign” material; or simply by hydrolysis. Examples of biodegradable materials are polymers such as Polydioxanone (PDO), Polycaprolactone (PCL), Polylactic acid (PLA), Polyglycolic acid (PGA), Adipic acid, PEG and glutamic acid.

The term “shape memory materials” refers hereinafter to materials which can “remember” there original geometry. After a sample of shape memory materials has been deformed from its original geometry, it regains its original geometry by itself during heating (one-way effect) or, at higher ambient temperatures, simply during unloading (pseudo-elasticity or superelasticity). The thermally induced shape-memory effect has been described for different material classes: polymers, such as polyurethanes, poly(styrene-block-butadiene), Polydioxanone and polynorbornene, metallic alloys, such as copper-zinc-aluminium-nickel, copper-aluminium-nickel, and nickel-titanium (NiTi) alloys.

The term ‘implantable’ refers to the property of an object wherein said object can be introduced into e.g. the human body at a great distance from the location wherein the implanting device enters the body. Arthroscopic techniques are often used as are those used for implanting stents and the like.

The term “reconstructing” refers to a process of building anew something which has broken, such as a bone. Reconstructing a broken bone could involve fixing two pieces of bone in place such that the two bone halves can grow back together.

The term “remolding” refers to a process of reshaping, in particular reshaping skin and/or muscle structure. Thus cosmetic surgery for instance often involves a process of remolding the face or other body parts.

The term ‘activated’ refers to an inflated, deflated, expanded, contracted, or otherwise non-equilibrium state of an object.

For example, a spring that is stretched, such that it is not at its equilibrium position, is considered for the purposes of this document to be activated.

Another example of an activated element is one that is filled with a liquid to reach an inflated or filled state as described in the definitions of inflatable or finable elements above.

The term ‘inactivated’ refers to a rest or equilibrium state of an object. For example, a spring that is not stretched, such that it is at its equilibrium position, is considered for the purposes of this document to be inactivated.

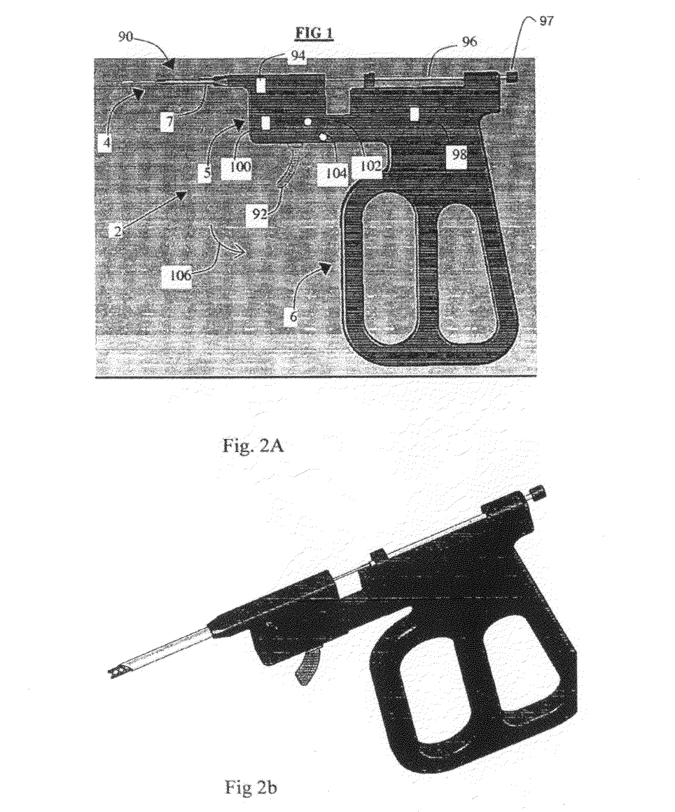

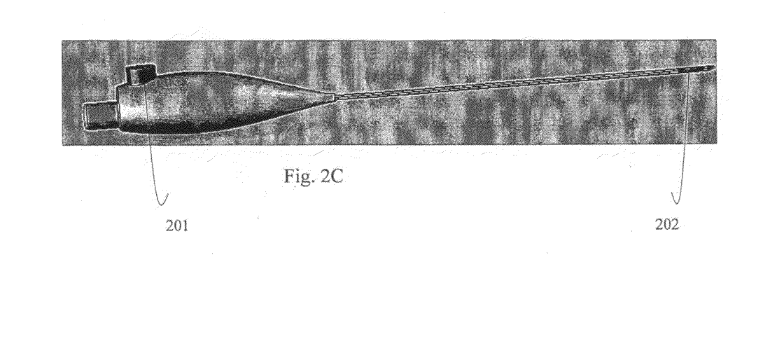

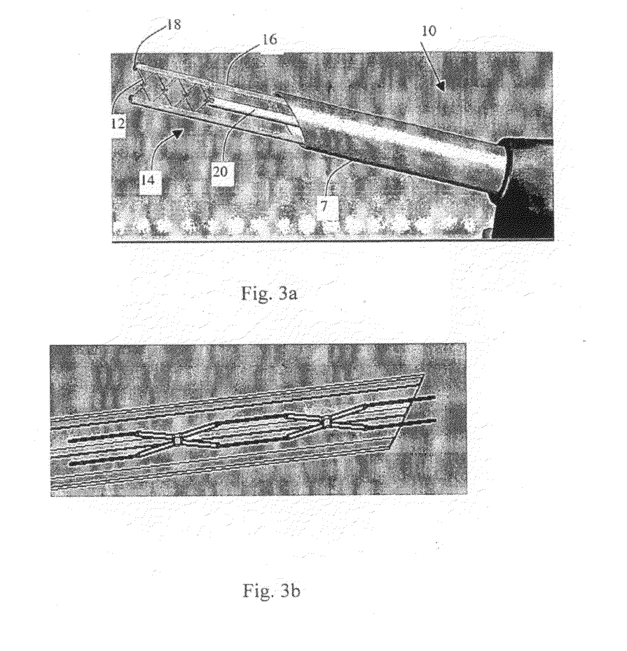

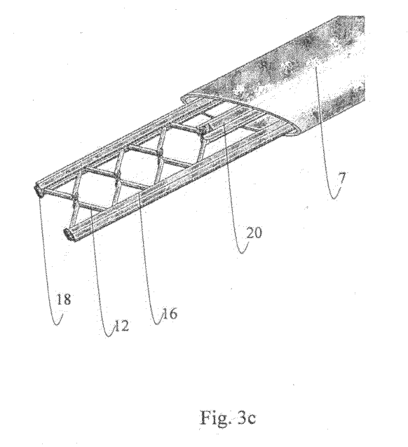

Reference is now made to FIG. 2a showing conducting tool 2 incorporating spring magazine 4 loaded with springs, according to a preferred embodiment of the present invention. Conducting tool 2 has body 5 and gripping handle 6. Tubular part 7 extends to a predefined length from the distal end of body 7. Spring magazine 4 loaded with one or more springs extends outwards from the interior lumen tubular part 7. The conducting tool is used to introduce a plurality of springs into specific locations and release them as needed. Tubular part 7 and spring magazine 4 including several internal parts, namely, slides, stoppers, hollow shaft and pulleys, (not visible), constitute together operational unit 90. Operational unit 90 is mountable into body 5 by an operator in an operating room prior to the surgical operation. Rotating lever 92 provides for either simultaneously or independently proximally pulling and distally pushing the tubular part 7 and the magazine respectively. Changing the operation of the device from independent to simultaneous movement is effected by rotating lever 92 perpendicular to the plane of the device. Thus the lever would extend out of the plane of the figure either towards the reader, or away from the reader. Such simultaneous or independent translations are carried out whilst body 5 and handle 6 are fixed. The operator moves the conducting tool only when he or she wishes to change from one target location to another. The exact location in which a spring is placed is located at the distal end of spring magazine 4. Lever 96 connected to a suitable gear mechanism at its distal end provides for opening releasing or confining the springs. This is accomplished by means of a pair of stoppers located at the end of spring magazine 4. (These stoppers are more clearly shown in FIG. 3). These openings and closings are accomplished by distally pushing the button 97 attached at the proximal end of lever 96. Mechanical indicator 98 ascends out of the plane of the paper (towards the reader) when the stoppers are opened and descends inwards when the stoppers are at a closed position. Pushing lever 100 provides for transferring the rotational lever 92 from a position in which it provides for moving the magazine 4 and the tubular part 7 to a position in which it provides for rotating the pulleys and stretching the sutures. This is accomplished by pushing lever 100 from the side above the plane of the paper to the side behind it. Push button 102 provides for momentarily releasing the pull exerted by both pulleys on the respective sutures thereby loosening them as long as push button 102 is being pressed by the operator. When the operator releases push button 102 the pulleys are automatically rotated to stretch the sutures at a predefined tension. This can be accomplished by various means such as by pulling the pulleys by means of a biasing spring stretched to a certain extent. Push button 104 provides for manually stretching both sutures by rotating lever 92 in the direction of arrow 106 when push button 104 is held pressed by the operator. In this state, rotating lever 92 automatically returns back (in a direction opposing arrow 106) when released. Another gear mechanism located within body 5, not shown, provides for changing the relation between the magnitude of the rotational angles of lever 92 to the tension forces applied onto the sutures.

In FIG. 2B another view of this introducer is shown.

In FIG. 2C another possible introducing element is shown. The actuating button 201 provides the function of releasing an anchoring element 202 from the distal end of the device.

Reference is now made to FIG. 3a, which is an isometric view of the distal portion of conducting tool 10 (referred to as tubular part 7 in FIG. 2). One or more implantable medical device (as will be discussed later on), such as implantable medical device 12, are disposed within spring magazine 14. The implantable medical device are successively arranged between slides 16 as shown. The implantable medical device are supported by the slides and are held in place by being compressed between stoppers 18 and the end of shaft 20. Optionally, two sutures are threaded through the hollow arms of the successive implantable medical device. The terminal ends of both sutures are respectively tied to, or internally locked within, the hollow arms of implantable medical device 12, which is the outermost spring in the line. The other terminal ends of these sutures are tied to a pair of pulleys, not shown. Both sutures pass through the lumen of shaft 20 and a segment of each suture is further wound around its respective pulley. Stoppers 18 are movable between two positions of which one is a closed position, and the other is an open position that allows the implantable medical device to be released from the spring magazine 14. Moving stoppers 18 between these positions is effected by means of two dedicated shafts respectively disposed within grooves at the inner faces of slides 16 and a lever (not shown) connected to a gear mechanism disposed within the body of conducting tool 10. The gear mechanism ultimately communicates with the release button 97 (of FIG. 2). Another pair of similar stoppers (not shown) is internally disposed, each stopper within the groove of its respective slide, adjacent to spring 12 confining the movement thereof inwards. When the two pairs of stoppers are simultaneously at a closed position, the distal-most implantable medical device 12 (and all other implantable medical device behind it) disposed within implantable medical device magazine 14 are internally locked between both slides 16 and shaft 20. When both pairs of stoppers are simultaneously at an open position the implantable medical devices can be successively pushed along slides 16 and out of implantable medical device magazine 14. The tubular part 7 in which the magazine 14 is mounted possibly can have an oval shape with optionally beveled end, as in the figure. The major axis of its elliptic cross section is preferably small, such as 3 millimetres length. The length of the other axis of the ellipse is of a few tenths of millimetres. Therefore the oval tube can be conveniently inserted into a tissue of a patient up to a significant depth that is limited by the surface of the body of the conducting tool.

FIG. 3b shows another possible example of this distal portion of the introducer.

FIG. 3c shows a closer view of a possible example of the distal portion of the introducer with stoppers 18, distal-most implantable medical device 12, slides 16, shaft 20, and tubular part 7.

FIG. 3d shows another embodiment of the introducer wherein the anchoring elements are threaded by a suture 301 passing through the centers of the implantable medical devices. The pulling element 302 pulls each of the implantable medical devices out of the end of the magazine when desired, by means of actuator within the introducer ‘gun’, not visible in this figure.

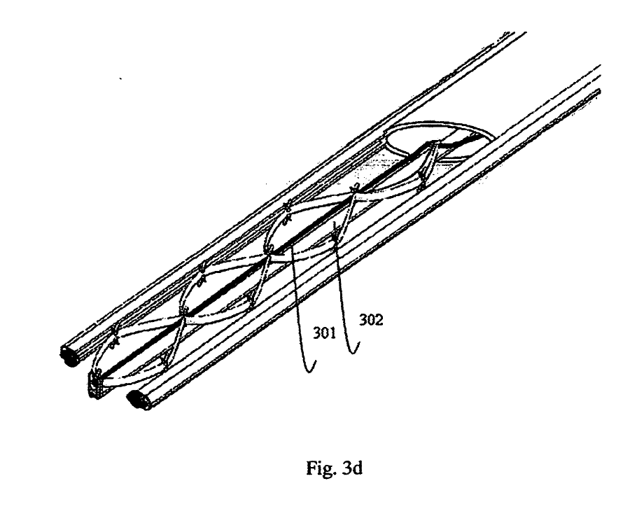

With reference to FIG. 4 one embodiment of anchoring elements within an introducer is illustrated. The implantable medical device 401 are held within the walls of the introducer 403 in a flexed position. They are flexed by the spring elements 402, which are held under compression by the introducer 403.

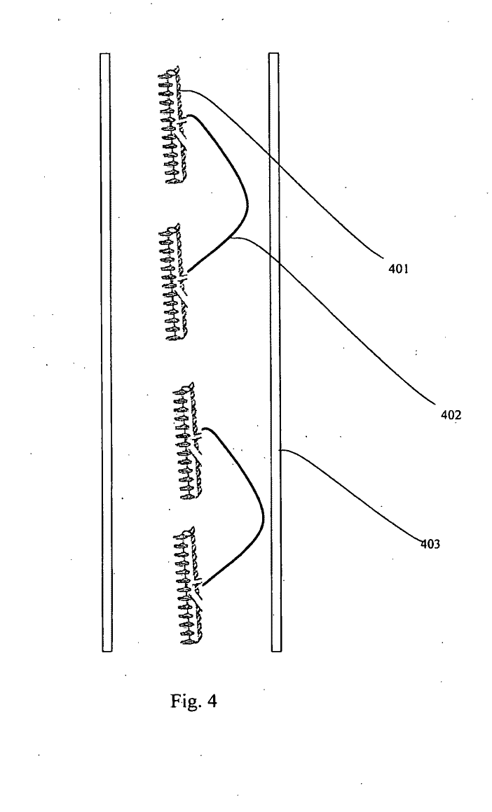

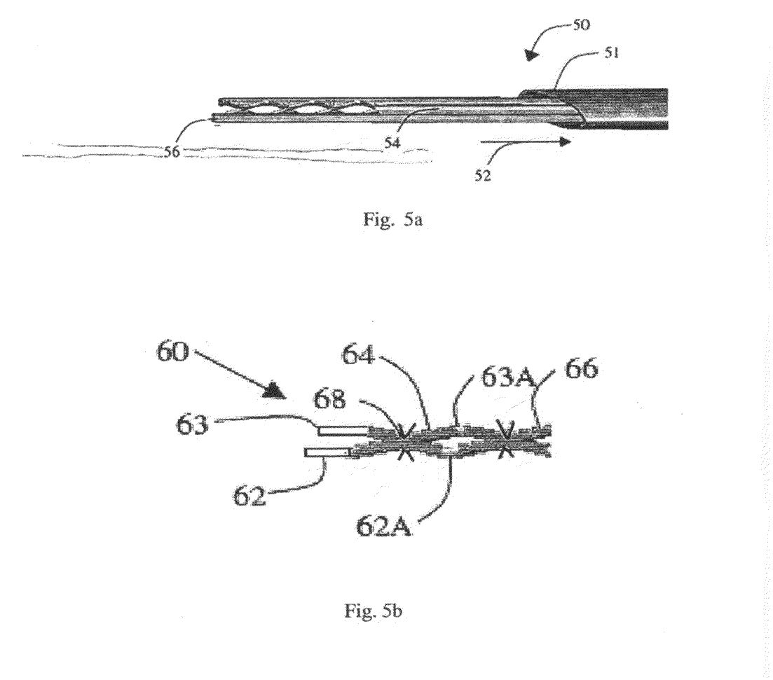

Reference is now made to FIG. 5A wherein a segment of conducting tool 50 is shown. To release an implantable medical device from the magazine, relative motion is provided between shaft 54 and tubular part 51. This may be accomplished by pulling tubular part 51 along the direction of arrow 52 and/or pushing shaft 54 in the opposite direction. These operations are performed when stoppers 56 are in their open configuration, allowing the springs to escape out the distal end of the device. It should be appreciated that a plurality of individual springs can be repeatedly disposed and or embedded into the targeted tissue during a single procedure, by the same conducting tool. Optionally the springs are threaded with sutures prior to being loaded into the spring magazine. Reference is now made to FIG. 5B, showing a segment 60 of a series of implantable medical devices interconnected by two sutures and embedded within a tissue. Sutures 62 and 63 are threaded through the lumens of respective arms of all the implantable medical devices. Both sutures are automatically clamped and locked by respective catchers within each of the embedded springs when released into its rest position. These sutures are loosely held and/or tightly stretched repeatedly during the entire process by means of the pulleys of the conducting tool. (One end of each suture is tied to, or locked within, the outermost implantable medical devices of the magazine, the other end is tied to the pulley and a segment of each suture is winded around its respective pulley.) Assuming that the placement and release of an implantable medical device 64 has been properly accomplished; sutures 62 and 63 are stretched between the catchers respectively disposed within implantable medical device 64 and the preceding implantable medical device, not shown, of this series; implantable medical devices 66 just slidingly emerges out of the magazine, not shown, and along the respective segments 62A and 63A of both sutures, which are loosen at this stage. Following the placement of implantable medical devices 66 prior to releasing it the operator synchronously stretches segment 62A and 63A up to a predefined level. It should be appreciated that by means of the independent control over the tension between any two successive springs provided by the invention, arbitrarily complex topological reconstructions can be accomplished.

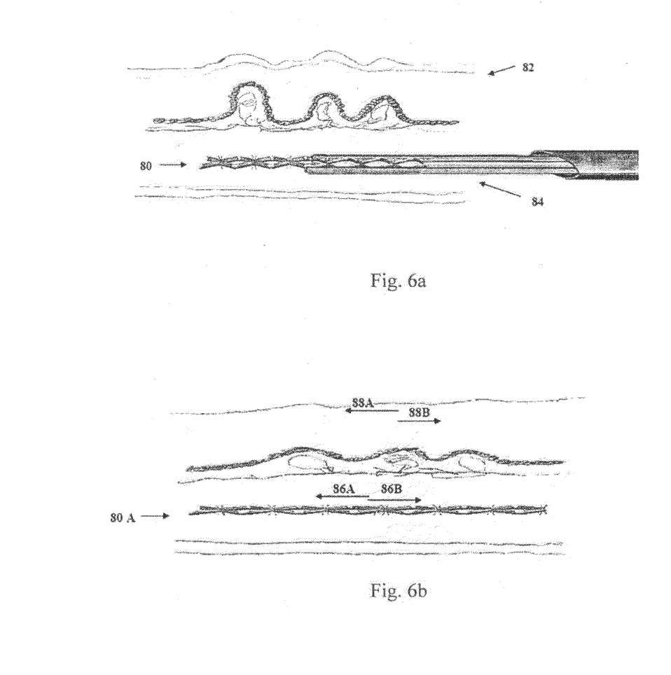

Reference is now made to FIGS. 6a, 6b, wherein embedding of implantable medical devices 80 in tissue 82 according to a preferred embodiment of the present invention is schematically shown implantable medical devices 80 is released from magazine 84 in its contracted state and subsequently extends longitudinally in directions of arrows 86A and 86B to elongated state 80A; thereby stretching the skin surface in directions of arrows 88A and 88B and thus flattening and smoothing the wrinkle as shown in FIG. 6B.

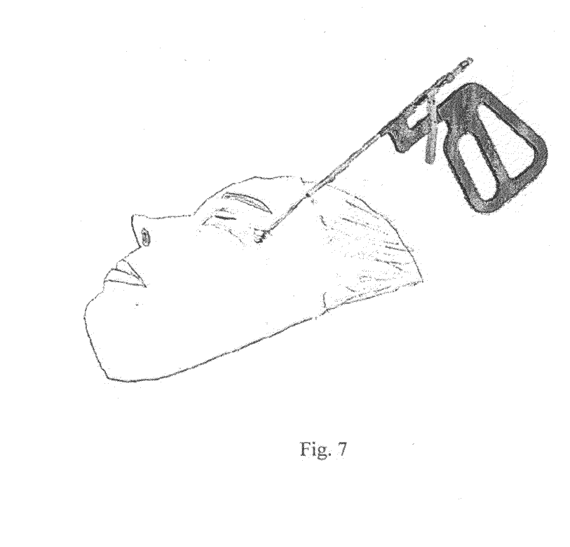

The tubular part of the conduction tool of the invention is introduced under the skin surface, as is shown in FIG. 7 to which reference is now made. The introduction is preferably but not necessarily made at the hairline, to conceal the point of entry. The distal end of the conducting tool is advanced under the skin surface to the targeted site. The spring magazine is in its contracted state at this time. The length of a desired stretch of the skin is preferably measured prior to the operation. This measure can translated to a level of stress applied on the springs prior to their loading into the spring magazine. The translation may be accomplished by means of prior experience, calibration tables, direct calculation, or the like.

It is within the scope of the present invention that the implantable medical devices be made of any elastic materials, such as stainless steel, plastic resins that are biocompatible and/or are coated with biocompatible materials, or materials typically used for producing stents or cogs typically providing for anchoring sutures. Materials capable of changing their elasticity by an internal molecular restructuring such as by externally heating them are applicable as well. The implantable medical devices are attachable to the tissues of a patient due to their geometrical shape, their elasticity, and/or optional spikes or cogs extending from their surfaces. Optionally the implantable medical devices are coated with suitable chemicals or drugs, such as botulinum toxin, antibiotic agents and/or growth factors, prior to their disposal within a patient tissue.

The implantable medical devices can be prepared form a variety of biomaterials substances, these can serve as either a constructive material or as a coating layer, or as a combination or as a composite and functional graded materials. These may include the following types: polymers (Bioabsorbable and durable: synthetic, and natural derived ones), metals (and different metals alloys), and ceramics. The polymer can be selected from a group consisting durable polymers, both synthetic and natural occurring materials including polyethylene, polypropylene, polyurethanes, poly (methyl metacrylate), polycarbonates, and silicone rubber. Biodegradable polymers, synthetic and natural occurring materials including polyalkylene esters, polylactic acid and its co-polymers, polyvinyl esters, polyvinyl alcohol, polyanhydrides, and polycarbonates.

The implantable medical devices can be prepared from metals such as Stainless steel, CoCr, Titanium, shape memory alloys.

The implantable medical devices can be prepared from ceramics such as hydroxyapaptite, bioactive glass, alumina, and zirconia.

The implantable medical devices can be prepared from Composite materials and functional graded materials such as a combination of various polymers and/or, metals, and/or ceramics.

Functional graded materials made: of either of various polymers and/or, metals, and/or ceramics that have gradually change in materials properties including crystallinity ratio, porosity level, and so on.

The implantable medical devices can be prepared from bioactive coatings such as proteins, growth factors, antigens, carbon like diamond, carbon, hyaluronic acid, collagen, silver, and gold.

The implantable medical devices are independently embedded in a targeted zone according to the method of the present invention. Alternatively a number of interconnected springs are successively embedded within a tissue, such that stretched sutures connect between adjacent springs.

According to one embodiment of the present invention an implantable medical device adapted for use in surgical operations. The device being implantable in a predetermined body portion to be treated. The device comprises (a) a hollow element having a biocompatible outer surface, said hollow element is characterised by an inactivated position and a plurality of activated positions; (b) a plurality of protrusions coupled to said outer surface, adapted to anchor said hollow element to said body portion to be treated; and, (c) coupling means accommodated within said hollow element, for imparting mechanical tension on said element.

According to another embodiment the shape of said element is selected from a group consisting of: zigzag shaped, square shape, C shape, of regular or irregular spiral, lumen, pipe, crescent, linear, bagel-like (annular), or star-like cross section, planar or proximally planar shaped barriers of any size, shape or type, 3D configurations, and multi-dimensional configurations comprising a plurality of regular or irregular two or more 2D planes and/or 3D spatial members interlinked together to form a continuous member.

It should be emphasized that the device can be anchored simply by applying outside pressure on the anchoring means. The pressure can be applied by the surgeon.

According to another embodiment the element is selected from a group consisting a spring, an inflatable element, fillable element.

According to another embodiment the element additionally comprises sealing means.

According to another embodiment the surgical operations are selected from a group consisting of cosmetic surgeries, especially for remoulding and/or reconstructing both soft and/or hard tissues, repairing apertures in soft biological tissue or any combination thereof.

According to another embodiment the implantable medical device is useful in cosmetic medical procedure selected from a group consisting of wrinkles removal, face lifting, or in medical procedure selected from a group consisting of intensive and/or immediate care for repairing apertures, or in medical procedure for repair aperture in hard biological tissue selected from a group consisting of orthopaedic surgeries, dental surgeries, soft and hard tissues reattachments.

According to another embodiment the elements are made of material selected from a group consisting of shape memory materials, biodegradable materials, biocompatible materials or any combination thereof.

It is within the scope of the present invention that the implantable medical devices and/or the element within the implantable medical device can be made of any elastic materials, such as stainless steel, plastic resins that are biocompatible and/or are coated with biocompatible materials, or materials typically used for producing stents or cogs typically providing for anchoring sutures. Materials capable of changing their elasticity by an internal molecular restructuring such as by externally heating them are applicable as well. The implantable medical devices are attachable to the tissues of a patient due to their geometrical shape, their elasticity, and/or optional spikes or cogs extending from their surfaces. Optionally the implantable medical devices are coated with suitable chemicals or drugs, such as botulinum toxin, antibiotic agents and/or growth factors, prior to their disposal within a patient tissue.

The implantable medical devices can be prepared form a variety of biomaterials substances, these can serve as either a constructive material or as a coating layer, or as a combination or as a composite and functional graded materials. These may include the following types: polymers (Bioabsorbable and durable: synthetic, and natural derived ones), metals (and different metals alloys), and ceramics. The polymer can be selected from a group consisting durable polymers, both synthetic and natural occurring materials including polyethylene, polypropylene, polyurethanes, poly (methyl metacrylate), polycarbonates, and silicone rubber. Biodegradable polymers, synthetic and natural occurring materials including polyalkylene esters, polylactic acid and its co-polymers, polyvinyl esters, polyvinyl alcohol, polyanhydrides, and polycarbonates.

The implantable medical devices can be prepared from metals such as Stainless steel, CoCr, Titanium, shape memory alloys. Alternatively, the implantable medical devices can be prepared from ceramics such as hydroxyapaptite, bioactive glass, alumina, and zirconia. Alternatively, the implantable medical devices can be prepared from Composite materials and functional graded materials such as a combination of various polymers and/or, metals, and/or ceramics. Functional graded materials made of either of various polymers and/or, metals, and/or ceramics that have gradually change in materials properties including crystallinity ratio, porosity level, and so on. Still Alternatively, the implantable medical devices can be prepared from bioactive coatings such as proteins, growth factors, antigens, carbon like diamond, carbon, hyaluronic acid, collagen, silver, and gold.

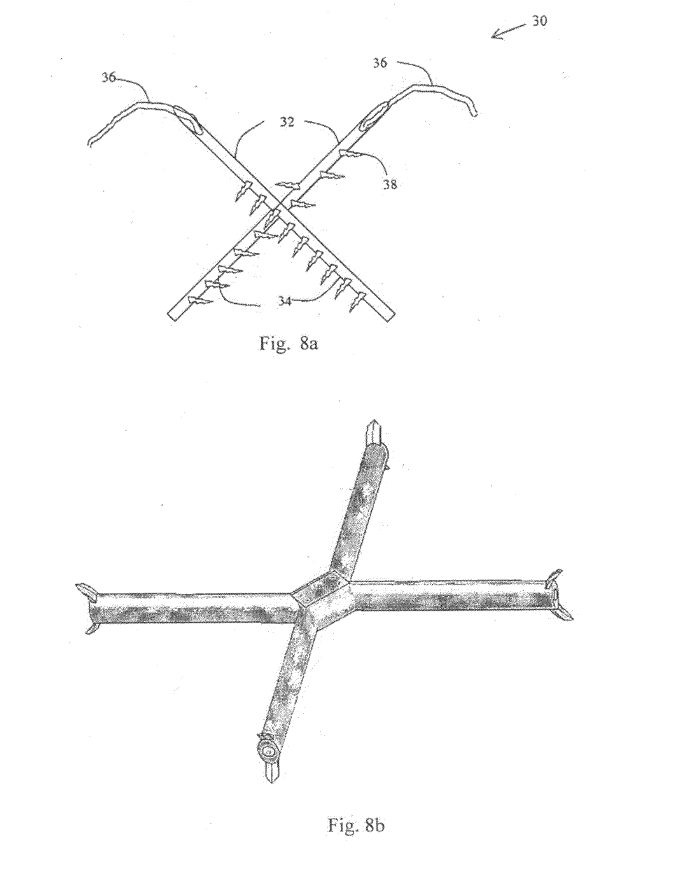

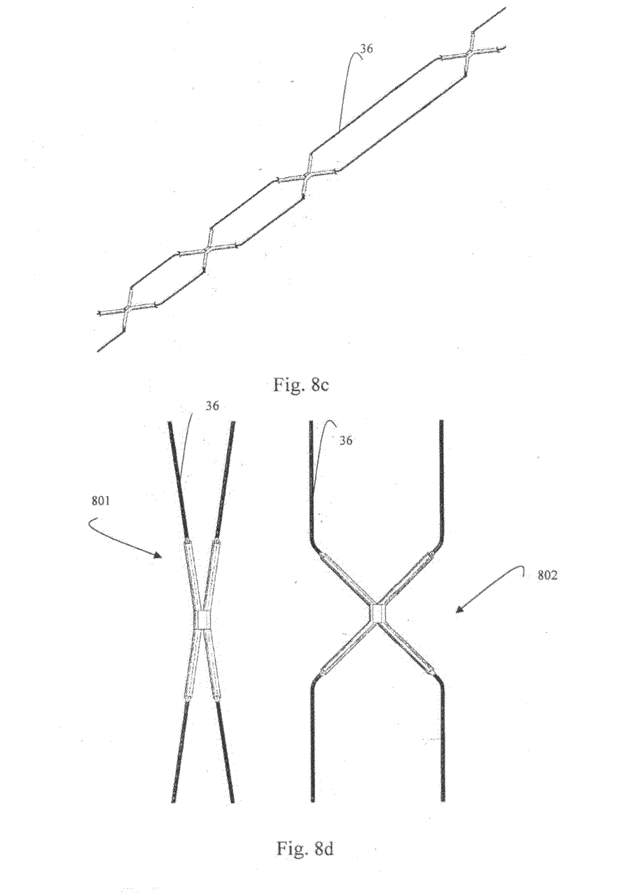

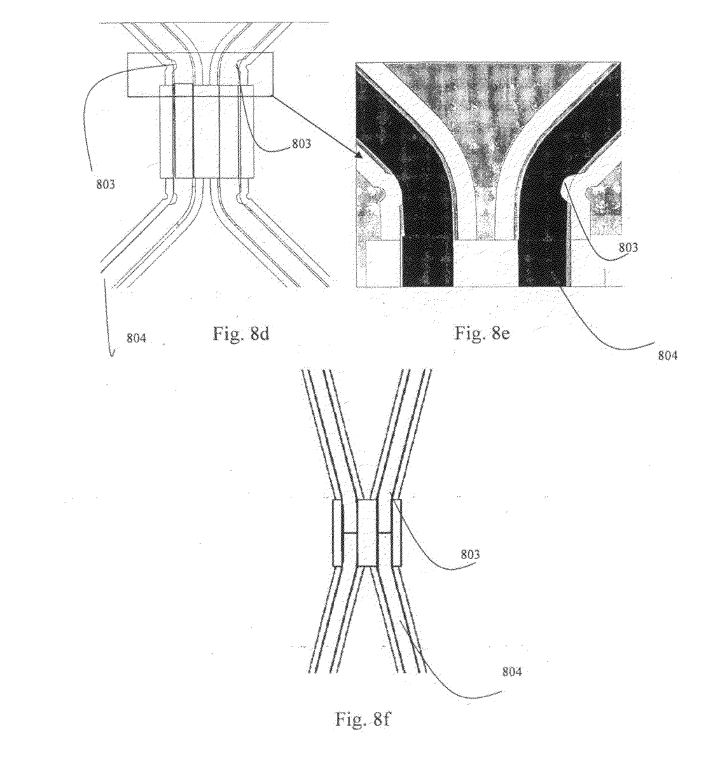

Reference is now made to FIGS. 8A and 8B which illustrate the above mentioned embodiment. FIGS. 8A and 8B illustrate two exemplary implantable medical device 30 shaped like the letter “X” has two pairs of hollow arms respectively indicated by the numerals 32 and 34. Suture 36 is optionally threaded trough the lumen of both arms 32. A second optional suture, not shown, is similarly threaded through the pair of arms 34. A catcher, not shown, internally disposed at the junction where the arms of both pairs are internally connected provides for locking both sutures when implantable medical device 30 is released to its rest position. Spikes or cogs, such as cog 38, extend, optionally resiliently, from the surfaces of the arms laterally pointing towards all directions. The cogs are respectively bent whilst the implantable medical device is enclosed within the walls of the oval tube. The bent cogs are sprung to their respective rest positions when released out of the oval tube. Each arm of implantable medical device 30 can be independently bent, pulled, stressed and/or twisted. The geometrical shapes of different arms of a implantable medical device need not be, the same. For example both arms of pair 32 can be curved out of the plane of the paper in the same or opposite directions and in the same or in different curvature angles. In FIG. 8B the cogs extend from the ends of the arms. Each arm can be curved having a respective curvature angle. Similarly two optional sutures can be respectively threaded through the hollow arms. This is illustrated in FIG. 8c where the sutures 36 can be seen traveling through the implantable medical device bodies and connecting each implantable medical device to its neighbor. Catchers internally disposed at the respective junctions where both arms of a pair are internally connected (not shown) provide for locking the arms when implantable medical device 40 is at rest position. The inactivated and activated states of one of the implantable medical devices is shown in FIG. 8d where the implantable medical device is kept in the activated state 801 while in the introducer, and then relaxes to the inactivated state 802 when released from the introducer. In FIG. 8d-f the suture locking mechanism is shown. This is a mechanism whereby the suture is locked into place when the anchoring element is released into its activated state. With reference to FIGS. 8d,e one sees the projection 803 that is pressed into the suture body 804, jamming the suture into the anchor element wall and thereby locking the suture into place. In FIG. 8f the anchoring element (i.e., the implantable medical device) is in its compressed state as when inside the magazine of the introducer. In this state the projection 803 no longer projects into the suture 804 and the suture is therefore able to be freely pulled through the anchoring element body.

The arms of a pair of any of the implantable medical devices described above can be disposed such that the angle between both arms can be closed or opened up to respective predefined extents. The implantable medical devices are bent, twisted, pulled and/or compressed into planar shapes when the implantable medical devices are inserted into the grooves of respective slides of an implantable medical device magazine. When a implantable medical device is completely released out of the slides it will tend to return towards its equilibrium shape (its rest state), modified of course by various stresses applied by the tissues in which the implantable medical device is embedded. A plurality of implantable medical devices can be threaded with the sutures, and each implantable medical device can be released at a designated zone. When the suture is pulled the tissue changes its contour by pulling the implantable medical devices towards one another. The end of the suture is then affixed to a certain area preferably under the hairline.

The present invention also provides a method for treating tissues in a predetermined body portion during surgical operations. The method comprising steps selected inter alia from (a) providing at least one implantable medical device comprises (i) a hollow element having a biocompatible outer surface, said hollow element is characterised by an inactivated position and a plurality of activated positions; (ii) a plurality of protrusions coupled to said outer surface, adapted to anchor said hollow element to said body portion to be treated; and, (iii) coupling means accommodated within said hollow element, for imparting mechanical tension on said element; (b) threading said coupling means through said hollow element; (c) incorporating said threaded implantable medical device in said inactivated position within an introducer; (d) introducing said implantable medical device via said introducer into said body portion to be treated; (e) at least partially anchoring said implantable medical device to said tissue in said body portion to be treated via said plurality of protrusions; (f) imparting mechanical tension onto said tissue via said coupling means, thereby treating said tissue in said predetermined body portion.

The present invention provides another method for treating tissues in a predetermined body portion during surgical operations. The method comprising steps selected inter alia from (a) providing at least two implantable medical device, each of which comprises N a hollow element having a biocompatible outer surface, said hollow element is characterised by an inactivated position and a plurality of activated positions; (ii) a plurality of protrusions coupled to said outer surface, adapted to anchor said hollow element to said body portion to be treated; and, (iii) coupling means accommodated within said hollow element, for imparting mechanical tension on said element; (b) threading said coupling means through said hollow element thereby mechanically coupling pairs of said implantable medical device together; (c) incorporating said threaded implantable medical devices in said inactivated position within an introducer; (d) introducing at least one of said implantable medical device via said introducer into said body portion to be treated; (e) at least partially anchoring said implantable medical device to said tissue in said body portion to be treated via said plurality of protrusions; (f) introducing the second element via said introducer into said body portion to be treated; (g) at least partially anchoring said second element to said tissue in said body portion to be treated via said plurality of cogs; (h) altering the tension in said coupling means such that the distance between said pairs is altered and mechanical tension is imparted on said tissue, thereby treating said tissue in said predetermined body portion.

According to another embodiment of the present invention the methods additionally comprising step of repeating said steps of incorporating, introducing, anchoring and altering.

According to another embodiment of the present invention the methods the step of tensing said tissue is performed by altering in an arbitrary geometric manner said pairs by means of independently fixed tension between every connected pair of said elements.

According to another embodiment of the present invention the methods additionally comprising step of affixing each of said flexible coupling means to said tissue in said predetermined body portion.

According to another embodiment a fastener for use in surgical operations is provided fastener being implantable in a predetermined body portion to be treated, wherein said fastener comprises (a) at least two arm positioned at an angle A relatively to one another; (b) a rigid stopper adapted to fixate said at least two arms at said angle A; and, (c) a plurality of protrusions coupled to at least a portion of said arms, adapted to anchor said fastener to said tissue in said body portion to be treated.

According to another embodiment the surgical operations are selected from a group consisting of cosmetic surgeries, especially for remoulding and/or reconstructing both soft and/or hard tissues, repairing apertures in soft biological tissue or any combination thereof.

According to another embodiment the fastener is useful in cosmetic medical procedure selected from a group consisting of wrinkles removal, face lifting or in medical procedure selected from a group consisting of intensive and/or immediate care for repairing apertures or in medical procedure for repair of apertures in hard biological tissue selected from a group consisting of orthopaedic surgeries, dental surgeries, soft and hard tissues reattachments.

According to another embodiment, angle A is greater than about 0 and lower than about 180 degrees.

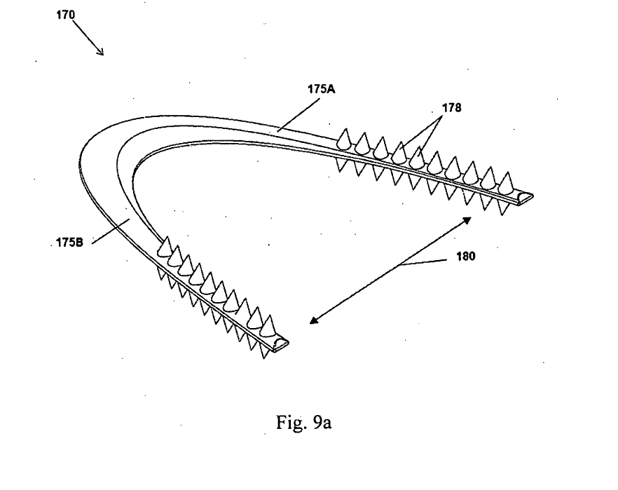

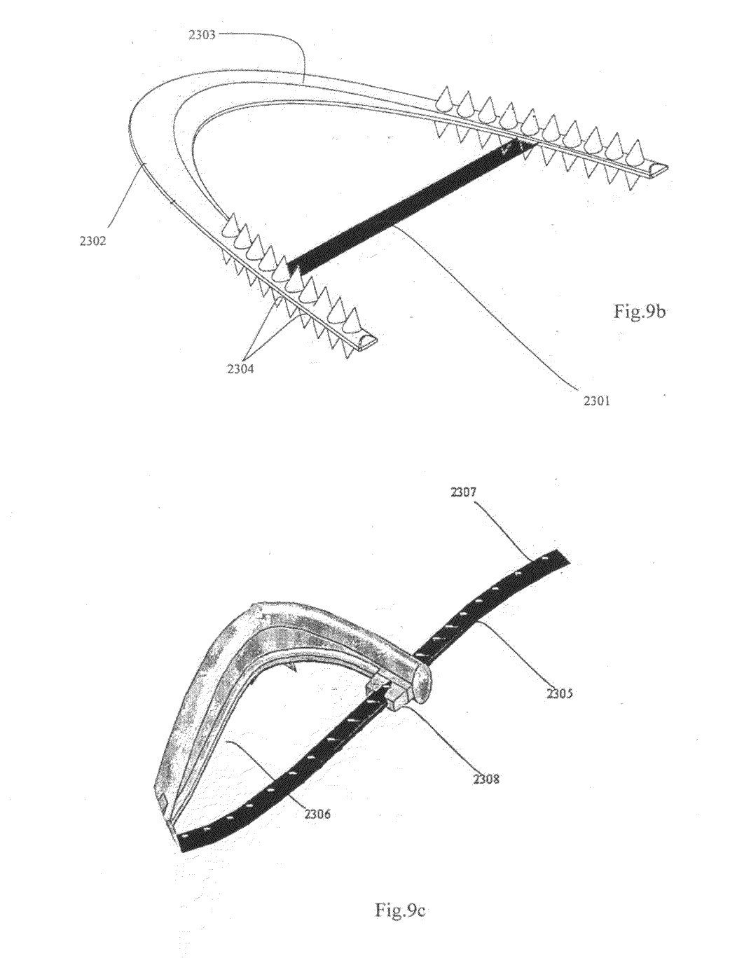

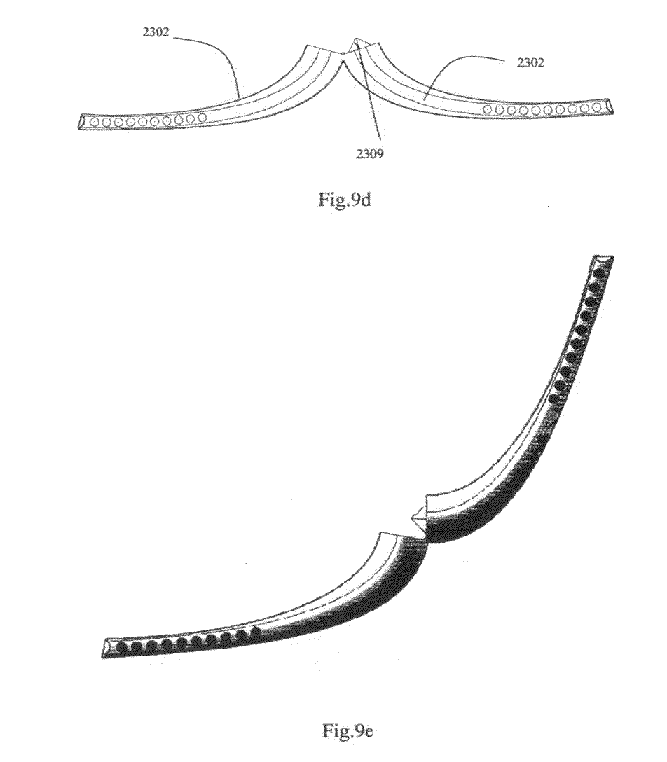

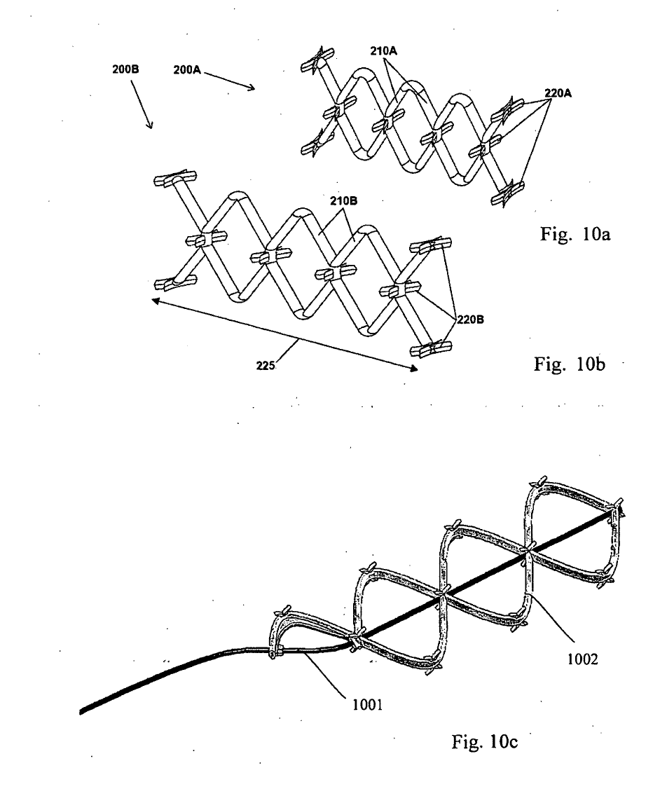

Reference is made now to FIG. 9a, showing another exemplary implantable medical device 170. Pair of arms 175A and 175B radiating from a common vertex, optionally with a hinge at their junction, or form an arcuate continuum with each other. Arms 175A and 175B are furnished with plurality of spikes or cogs 178. Implantable medical device 170 is being contained within the magazine of the conducting tool at the contracted state so that the distance or radial angle between arms 175A and 175B represented by arrow 180 is relatively smaller than at the rest position of implantable medical device 170. Upon disposing implantable medical device 170 from the magazine of the conducting tool the distance or radial angle between represented by arrow 180 is to increase and thereby to induce the desired aesthetic effect. Implantable medical device 170 can be beneficially employed for smoothing wrinkles and affixing tissue at the corner of the eye, particularly at the zygomatic eye corners.

With reference to FIG. 9b one embodiment of an anchoring element is illustrated. The implantable medical device can be opened or closed by the positioning rigid element 2301. The fastener comprises (a) at least two arms 2303 positioned at an angle A relatively to one another; (b) a rigid element 2301 adapted to fix said at least two arms at said angle A; and, (c) a plurality of cogs 2304 coupled to said arms, adapted to anchor said anchoring element to the tissue of a body portion to be treated.

In FIG. 9c an alternative embodiment is shown wherein a suture 2305 threading the anchoring device 2306 is provided with a plurality of ratchets 2307 that prevent the suture from releasing tension, by a ratching mechanism whereby once a ratchet passes through the locking mechanism 2308 it cannot pass back through it in the opposite direction.

In FIG. 9d an alternative embodiment is shown wherein the anchoring elements are provided with hinges 2309 to allow for a compressed state while stored in the introducer, and an expanded state when released into body tissues. In FIG. 9e the partially closed element is shown, after having been released from the introducer but before complete relaxation into the expanded C-shaped form.

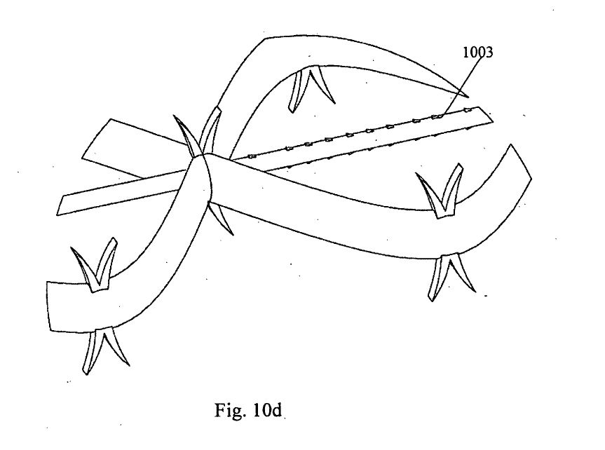

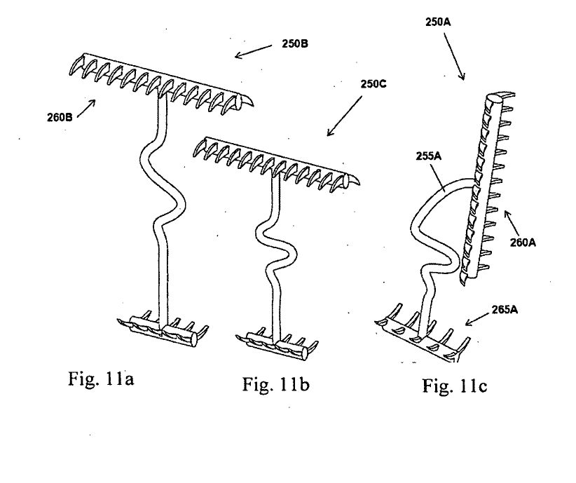

Reference is made now to FIGS. 10A and 109B, showing yet another exemplary implantable medical device 200A and 200B at contracted and rest states respectively. A plurality of interlocking elements 210A and 210B bear some structural and functional resemblance to a scissor-jack. Spikes or cogs 220A and 220B extend from the junctions, terminal ends or elsewhere on the surface of interlocking elements 210A and 210B. Implantable medical device 200A is being contained within the magazine of the conducting tool at the contracted state so that the length thereof is relatively smaller than at the rest position 210B represented by arrow 225. Upon disposing implantable medical device 200A from the magazine of the conducting tool implantable medical device extends in direction of arrow 225 to the rest position 210B and thereby induces the desired aesthetic effect. Such implantable medical devices can be beneficially employed for stretching a wrinkle in the following manner: the ends of the implantable medical device are affixed to the tissue on the opposite sides of the wrinkle, the implantable medical device is then allowed to extend longitudinally thereby stretching and flattening the wrinkle. In FIG. 10c a variation upon this design is shown wherein the suture 1001 is threaded between the implantable medical device elements 1002 as shown, allowing the entire assembly to be tensed or relaxed as a whole by pulling upon the suture to the desired degree of tension. In FIG. 10d a close up of one of the anchoring elements of a version of the device is shown. Here one sees that in this embodiment the suture has been provided with ratchets 1003 such that the suture will retain tension when released.

According to another embodiment of the present invention, an implantable medical device adapted for use in surgical operations is provided. The device being implantable in a predetermined body portion to be treated. The predetermined body portion is characterized by a first extremity and by a second extremity. The device comprises (a) at least one element having a body, a distal end, and a proximal end; said body being characterized by (i) at least one inactivated position; and, (ii) a plurality of activated positions; and, (b) at least one anchoring means coupled to said distal end of said element, adapted to anchor said distal end to said first extremity in said predetermined body portion; (c) at least one anchoring means coupled to said proximal end of said element, adapted to anchor said proximal end to said second extremity in said predetermined body portion.

The element is at least partially reconfigurable from said activated position to said inactivated position and/or from said plurality of inactivated positions to said plurality of activated positions such that the—distance between said first extremity and said second extremity is alterable.

It should be emphasized that the device can be anchored simply by applying outside pressure on the anchoring means. The pressure can be applied by the surgeon.

According to another embodiment the surgical operations are selected from a group consisting of cosmetic surgeries, especially for remoulding and/or reconstructing both soft and/or hard tissues, repairing apertures in soft biological tissue or any combination thereof. Furthermore, it can be useful in cosmetic medical procedure selected from a group consisting of wrinkles removal, face lifting. Moreover, it can be useful in medical procedure selected from a group consisting of intensive and/or immediate care for repairing apertures or in medical procedure for repair of apertures in hard biological tissue selected from a group consisting of orthopaedic surgeries, dental surgeries, soft and hard tissues reattachments.

According to another embodiment the at least two anchoring means are positioned at an angle A relatively to each other. The angle A is greater than about 0 degrees and lower than about 180 degrees, especially at an angle 180 degrees.

According to another embodiment the element is selected from a group consisting of a spring, an inflatable element, fillable element.

According to another embodiment the element additionally comprises sealing means.

According to another embodiment the element may be transformed by means selected from group consisting of: stretching, compression, inflation, deflation.

According to another embodiment the element is made of material selected from a group consisting of shape memory materials, biodegradable materials, biocompatible materials or any combination thereof.

It is within the scope of the present invention that the implantable medical devices and/or the element within the implantable medical device can be made of any elastic materials, such as stainless steel, plastic resins that are biocompatible and/or are coated with biocompatible materials, or materials typically used for producing stents or cogs typically providing for anchoring sutures. Materials capable of changing their elasticity by an internal molecular restructuring such as by externally heating them are applicable as well. The implantable medical devices are attachable to the tissues of a patient due to their geometrical shape, their elasticity, and/or optional spikes or cogs extending from their surfaces. Optionally the implantable medical devices are coated with suitable chemicals or drugs, such as botulinum toxin, antibiotic agents and/or growth factors, prior to their disposal within a patient tissue.

The implantable medical devices can be prepared form a variety of biomaterials substances, these can serve as either a constructive material or as a coating layer, or as a combination or as a composite and functional graded materials. These may include the following types: polymers (Bioabsorbable and durable: synthetic, and natural derived ones), metals (and different metals alloys), and ceramics. The polymer can be selected from a group consisting durable polymers, both synthetic and natural occurring materials including polyethylene, polypropylene, polyurethanes, poly (methyl metacrylate), polycarbonates, and silicone rubber. Biodegradable polymers, synthetic and natural occurring materials including polyalkylene esters, polylactic acid and its co-polymers, polyvinyl esters, polyvinyl alcohol, polyanhydrides, and polycarbonates.

The implantable medical devices can be prepared from metals such as Stainless steel, CoCr, Titanium, shape memory alloys. Alternatively, the implantable medical devices can be prepared from ceramics such as hydroxyapaptite, bioactive glass, alumina, and zirconia. Alternatively, the implantable medical devices can be prepared from Composite materials and functional graded materials such as a combination of various polymers and/or, metals, and/or ceramics. Functional graded materials made of either of various polymers and/or, metals, and/or ceramics that have gradually change in materials properties including crystallinity ratio, porosity level, and so on. Still Alternatively, the implantable medical devices can be prepared from bioactive coatings such as proteins, growth factors, antigens, carbon like diamond, carbon, hyaluronic acid, collagen, silver, and gold.

According to another embodiment the implantable medical device additionally comprising at least one anchoring means coupled to said body of at least one of said elements.

According to another embodiment the anchoring means are mechanically linked.

Reference is made now to FIGS. 11a, 11b and 11c, illustrating the above mentioned implantable medical device. FIGS. 11a, 11b and 11c illustrating the implantable medical device 250A, 250B and 250C at contracted, longitudinally stretched (both are different activated positions) and rest states (i.e., the inactivated position) respectively. Resilient flexible serpentine 255A (i.e., the element) interconnects between upper anchor 260A and lower anchor 265A that are furnished with spikes or cogs as shown. Resilient flexible serpentine 255A (i.e., the element) is being bended or twisted while contained in within the magazine of the conducting tool (i.e., introducer). Upon disposing lower anchor 265A of implantable medical device 250A from the magazine of conducting tool anchor 265A can be affixed to the tissue. Subsequently upper anchor 260A is released from the magazine and driven by the bias of flexible serpentine 255A into a predefined position relatively to lower anchor 265A. The implantable medical device is than stretched longitudinally as shown in FIG. 10B and upper anchor thereof 260B is also affixed to the tissue. The implantable medical device is then allowed to voluntarily contract into rest state 250C thereby inducing the desired aesthetic effect.

Alternatively, one may anchor the upper anchor 260A and the lower anchor 265A and only then reconfigure the resilient flexible serpentine 255A (i.e., the element) from an activated position to an inactivated position or from an inactivated position to an activated position. This may be enabled if the resilient flexible serpentine 255A is made of a self activated material such as shape memory material.

Such implantable medical device can be beneficially employed for stretching a wrinkle in the following manner: the ends of the implantable medical device are affixed to the tissue on the same side of the wrinkle, the implantable medical device is then allowed to longitudinally contact thereby gathering the tissue at one side of the wrinkle, thus flattening the wrinkle.

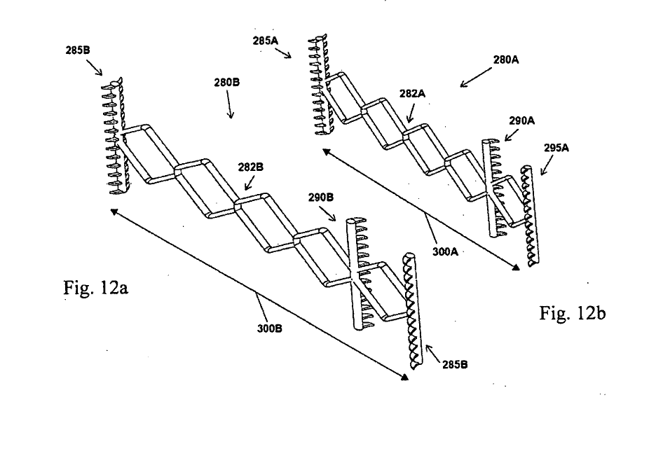

Reference is made now to FIGS. 12A and 12B, showing yet another exemplary implantable medical device 280A and 280B at contracted and rest states respectively. A wire-mesh framework 282A and 282B can be furnished with a plurality of anchors. The anchors are versatile and can be of different types; thus some anchors can be furnished with spikes or cogs bilaterally so that the spikes or cogs facing in to two different or opposite directions, such as an exemplary anchor 285A and 285B. Some anchors can be furnished with spikes or cogs unilaterally so that the spikes or cogs face solely in one direction, such as an exemplary anchor 290A, 290B, 295A and 295B, so as to selectively affix the respective anchor either the skin or the muscle tissue. Implantable medical device 280A is contained within the magazine of the conducting tool at the contracted state so that the length of wire-mesh framework 282A thereof represented by arrow 300A is relatively smaller than the length of wire-mesh framework 280B at rest position 280B represented by arrow 300B. Upon disposing implantable medical device 280A from the magazine of the conducting tool implantable medical device extends in direction of arrow 300B to the rest position 280B, or vice versa, and thereby inducing the desired aesthetic effect. Such a implantable medical device may be beneficially employed for stretching a wrinkle in the following manner: the bilateral anchor of the implantable medical device is affixed to the facial muscle tissue and the skin at the one side of the wrinkle. The implantable medical device is then released from the magazine and either one of its unilateral anchors or both of them can be respectively affixed to the facial muscle tissue and or the skin on the opposite side of the wrinkle. The implantable medical device is then allowed to extend longitudinally thereby stretching and flattening the wrinkle. Conversely, the implantable medical device may be used to induce contraction between the anchors at the different ends and can be beneficially employed for stretching a wrinkle in the following manner: the anchors at the different ends of the implantable medical device are affixed to the tissue on the same side of the wrinkle. The implantable medical device is then allowed to longitudinally contact, thereby gathering the tissue at one side of the wrinkle, thus flattening the wrinkle.

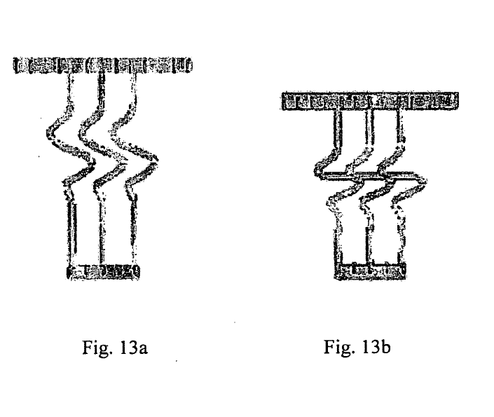

In FIGS. 13a and 13b further embodiments of the implantable medical devices are given. In said figure, an embodiment in which more than one flexible member is shown. This may enable to vary the degree of tension. FIG. 13a illustrates the implantable medical device in its stretched position (i.e., activated position) and, FIG. 13b illustrates the implantable medical device in its compressed position (i.e., activated position) or in its relaxed position (i.e., inactivated position).

The present invention also provides a method for treating tissues in a predetermined body portion during surgical operations, said predetermined body portion to be treated is characterized by a first extremity and a second extremity, said method comprising steps of (a) providing an implantable medical device comprising (i) at least one element having a body, a distal end, and a proximal end; said body is characterized by (i) at least one inactivated position; and, (ii) a plurality of activated positions; (ii) at least one anchoring means coupled to said distal end of said element; and, (iii) at least one anchoring means coupled to said proximal end of said element; (b) introducing said implantable medical device in said activated position or in said inactivated position to said body portion to be treated; (c) at least partially reversibly anchoring said distal end of said implantable medical device to said first extremity in said predetermined body portion via one of said anchoring means; (d) reconfiguring said body from said activated position to said inactivated position and/or from said inactivated position to said activated position; (e) at least partially reversibly anchoring said proximal end of said implantable medical device to said second extremity in said predetermined body portion via one of said anchoring means, thereby imparting tension and altering the distance between said first extremity and said second extremity thus, treating said tissue of said body portion.

The present invention also provides another method for treating tissues in a predetermined body portion during surgical operations, said method comprising steps of (a) providing an implantable medical device comprising (i) at least one element having a body, a distal end, and a proximal end; said body is characterized by (i) at least one inactivated position; and, (ii) a plurality of activated positions; (ii) at least one anchoring means coupled to said distal end of said element; and, (iii) at least one anchoring means coupled to said proximal end of said element; (b) introducing said implantable medical device in said activated position or in said uncativated position to said body portion to be treated; (c) at least partially reversibly anchoring said distal end of said implantable medical device to said first extremity in said predetermined body portion via one of said anchoring means; (d) at least partially reversibly anchoring said proximal end of said implantable medical device to said second extremity in said predetermined body portion via one of said anchoring means; and, (e) reconfiguring said body from said activated position to said inactivated position and/or from said inactivated position to said activated position; imparting tension and altering the distance between said first extremity and said second extremity thereby treating said tissue of said body portion.

According to another embodiment the methods additionally comprising step of releasing either one of said anchoring means.

According to another embodiment the methods additionally comprising the step of reconfiguring said at least one element from said activated position to said inactivated position and/or from said inactivated position to said activated position.

According to another embodiment of the present invention an implantable medical device adapted for use in surgical operations is provided. The device being implantable in a predetermined body portion to be treated. The device comprises (a) at least one stationary anchoring means; (b) at least one movable anchoring means in mechanical communication with said stationary anchoring means via at least one shaft; said movable anchoring means is adapted to reciprocally move along the longitudinal axis of said shaft; and, (c) at least one element having a body, a distal end and a proximal end; said body is characterized by at least one inactivated position and a plurality of activated positions; said element is coupled to said movable anchoring means at least one end of said element; said element is at least partially reconfigurable, from said activated position to said inactivated position and/or from said plurality of inactivated positions to said plurality of activated positions.

According to another embodiment the device additionally comprising means for activating said element, such that said body is reconfigured from said activated position to said inactivated position and/or from said inactivated position to said activated position.

It should be emphasized that the device can be anchored simply by applying outside pressure on the anchoring means. The pressure can be applied by the surgeon.

According to another embodiment, said means for activating is selected from a group consisting of inflating or deflating means, filling or withdrawing means, pressure, tension, force, heat, luminescence, change of Ph, application of magnetic field, application of electric field, voltage.

According to another embodiments the surgical operations are selected from a group consisting of cosmetic surgeries, especially for remoulding and/or reconstructing both soft and/or hard tissues, repairing apertures in soft biological tissue or any combination thereof. Furthermore, the implantable medical device may be useful in cosmetic medical procedure selected from a group consisting of wrinkles removal, face lifting, intensive and/or immediate care for repairing apertures, in repairing apertures in hard biological tissue selected from a group consisting of orthopaedic surgeries, dental surgeries, soft and hard tissues reattachments.

According to another embodiment the at least two anchoring means are positioned at an angle A relatively to each other.

According to another embodiment angle A is greater than about 0 degrees and lower than about 180 degrees, especially at an angle of 0 degrees.

According to another embodiment the element is selected from a group consisting a spring, an inflatable element, finable element.

According to another embodiment the element additionally comprises sealing means.

According to another embodiment the element is made of material selected from a group consisting of shape memory materials, biodegradable materials, biocompatible materials or any combination thereof.

It is within the scope of the present invention that the implantable medical devices and/or the element within the implantable medical device can be made of any elastic materials, such as stainless steel, plastic resins that are biocompatible and/or are coated with biocompatible materials, or materials typically used for producing stents or cogs typically providing for anchoring sutures. Materials capable of changing their elasticity by an internal molecular restructuring such as by externally heating them are applicable as well. The implantable medical devices are attachable to the tissues of a patient due to their geometrical shape, their elasticity, and/or optional spikes or cogs extending from their surfaces. Optionally the implantable medical devices are coated with suitable chemicals or drugs, such as botulinum toxin, antibiotic agents and/or growth factors, prior to their disposal within a patient tissue.

The implantable medical devices can be prepared form a variety of biomaterials substances, these can serve as either a constructive material or as a coating layer, or as a combination or as a composite and functional graded materials. These may include the following types: polymers (Bioabsorbable and durable: synthetic, and natural derived ones), metals (and different metals alloys), and ceramics. The polymer can be selected from a group consisting durable polymers, both synthetic and natural occurring materials including polyethylene, polypropylene, polyurethanes, poly (methyl metacrylate), polycarbonates, and silicone rubber. Biodegradable polymers, synthetic and natural occurring materials including polyalkylene esters, polylactic acid and its co-polymers, polyvinyl esters, polyvinyl alcohol, polyanhydrides, and polycarbonates.

The implantable medical devices can be prepared from metals such as Stainless steel, CoCr, Titanium, shape memory alloys. Alternatively, the implantable medical devices can be prepared from ceramics such as hydroxyapaptite, bioactive glass, alumina, and zirconia. Alternatively, the implantable medical devices can be prepared from Composite materials and functional graded materials such as a combination of various polymers and/or, metals, and/or ceramics. Functional graded materials made of either of various polymers and/or, metals, and/or ceramics that have gradually change in materials properties including crystallinity ratio, porosity level, and so on. Still Alternatively, the implantable medical devices can be prepared from bioactive coatings such as proteins, growth factors, antigens, carbon like diamond, carbon, hyaluronic acid, collagen, silver, and gold.

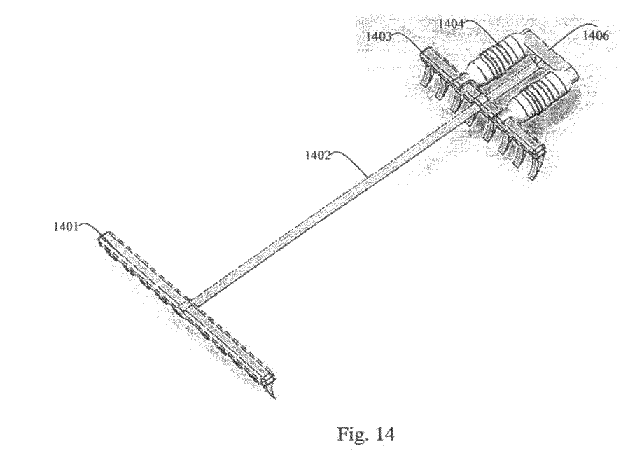

FIG. 14 illustrate the above mentioned embodiment. The anchor 1401 (i.e., the stationary anchoring means) is attached to rigid member 1402 (i.e., shaft). Second anchor 1403 (i.e., the movable anchoring means) is attached to the rigid member 1402 by means of inflatable pistons 1404 (i.e., the element), which are attached to the rigid member by coupling 1406. These pistons may be inflated or filled with liquid, or otherwise pressurized, causing anchor 1403 to move along rigid member 1402. Once the element 1403 has been placed above its desired position it is pressed into place, anchoring it into the tissue. Then, it is optionally that the positioning elements 1404, 1406 to be removed since the anchors 1401, 1403 are now in position and anchored. This allows for the minimal amount of material to be left in situ.

In FIG. 14b a similar embodiment of an anchoring and tensioning mechanism is given. The anchor elements 1401 and 1402 are, forced together or apart by means of collapsible member 1403, which is inflated by pressurizing, filling with liquid, or the like. This action forces the anchor ends 1401, 1402 apart, providing tension to the anchored tissue when anchored.

The present invention also provides a method for treating tissues in a predetermined body portion during surgical operations, said predetermined body portion to be treated is characterized by a first extremity and a second extremity. The method comprising steps selected inter alia from (a) providing an implantable medical device having (i) at least one stationary anchoring means; (ii) at least one movable anchoring means in mechanical communication with said stationary anchoring means via at least one shaft; and, (iii) at least one element having a body, a distal end and a proximal end, said body is characterized by at least one activated position and at least one inactivated position; (b) coupling said movable anchoring means to said element at said distal end or said proximal end; (c) inserting said implantable medical device in said activated position or in said inactivated position into said body portion to be treated; (d) at least partially reversibly anchoring said stationary anchoring means to said first extremity in said body portion to be treated; (e) at least partially reversibly anchoring said movable anchoring means to said second extremity in said body portion to be treated; and, (f) reversibly activating said body of said at least one element from said activated position to said inactivated position and/or from said inactivated position to said activated position such that said movable anchoring means is reciprocally moving along said shaft; thereby imparting tension and altering the distance between said first extremity and said second extremity thus, treating said tissue of said body portion.

According to another embodiment, the method additionally comprising step of releasing either one of said anchoring means.

According to another embodiment, the method additionally comprising the step of reconfiguring said at least one element from said activated position to said inactivated position and/or from said inactivated position to said activated position.

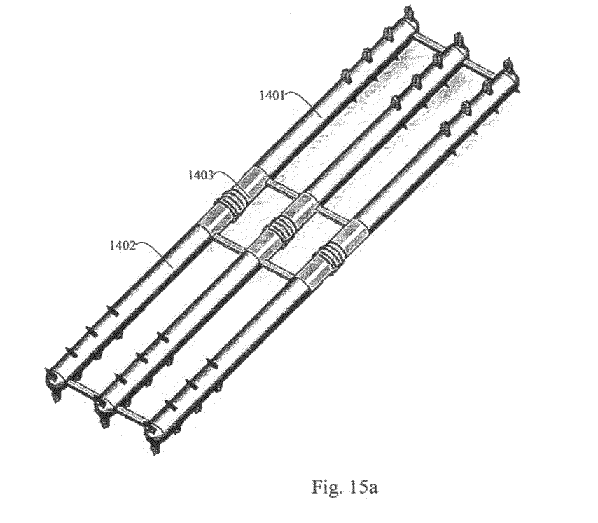

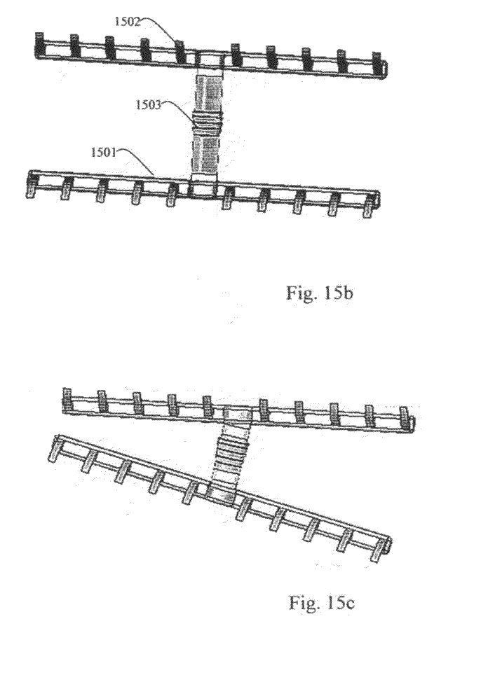

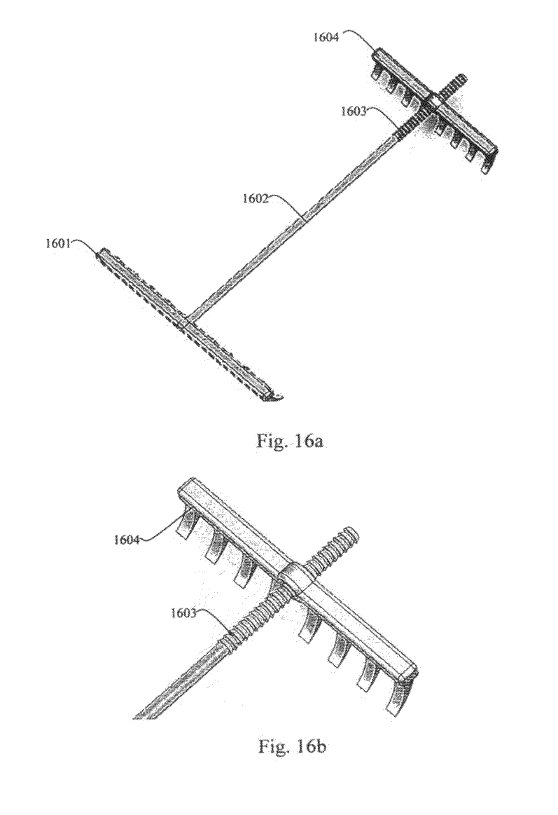

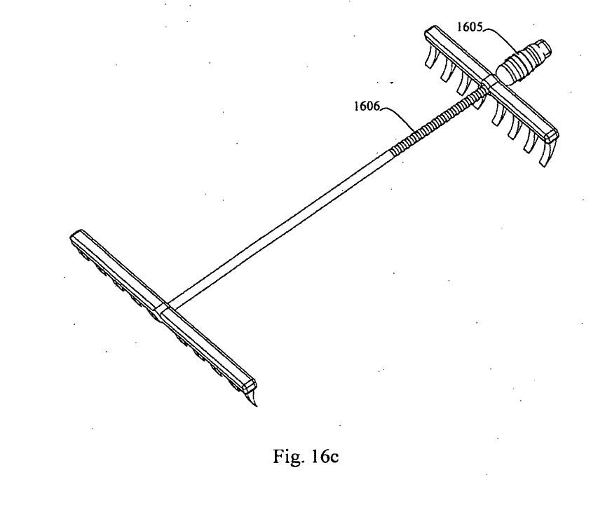

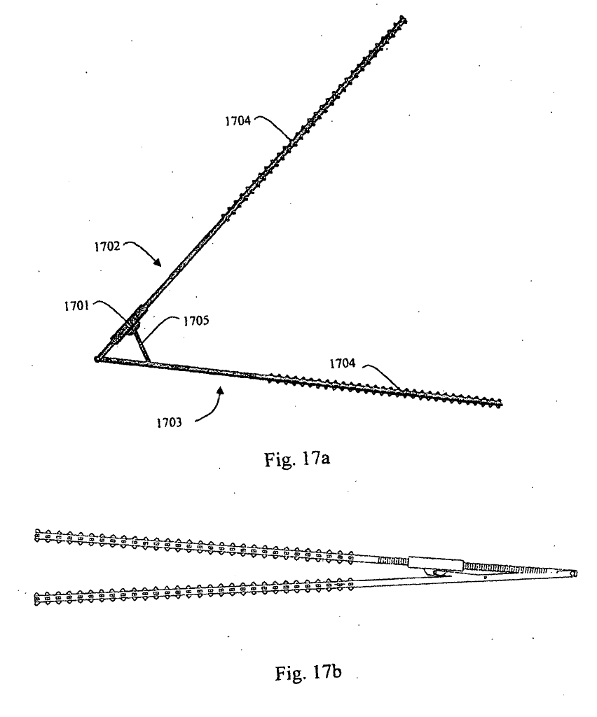

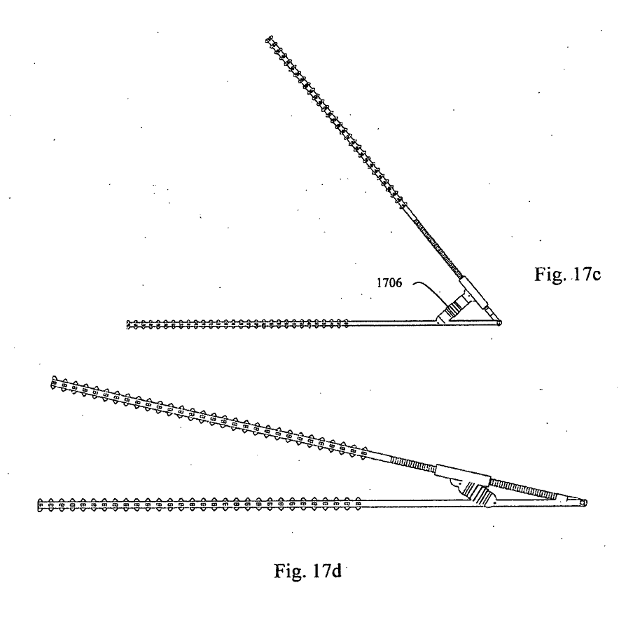

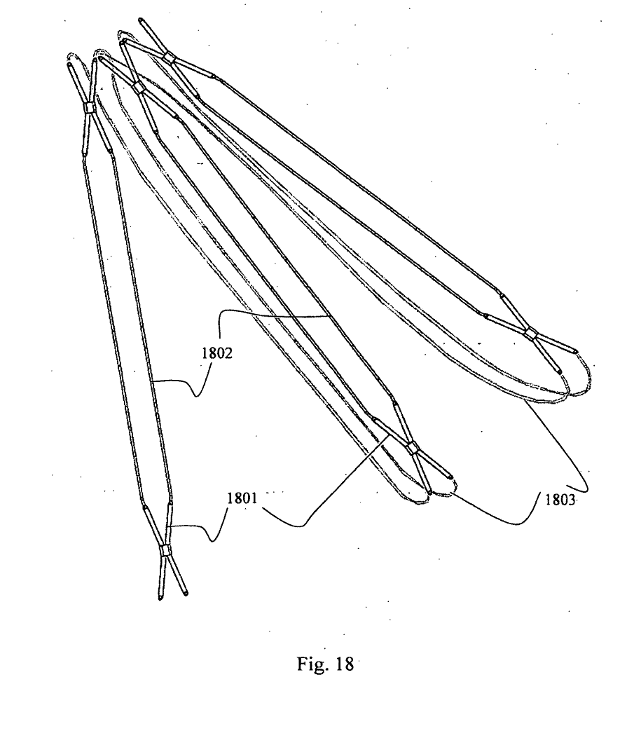

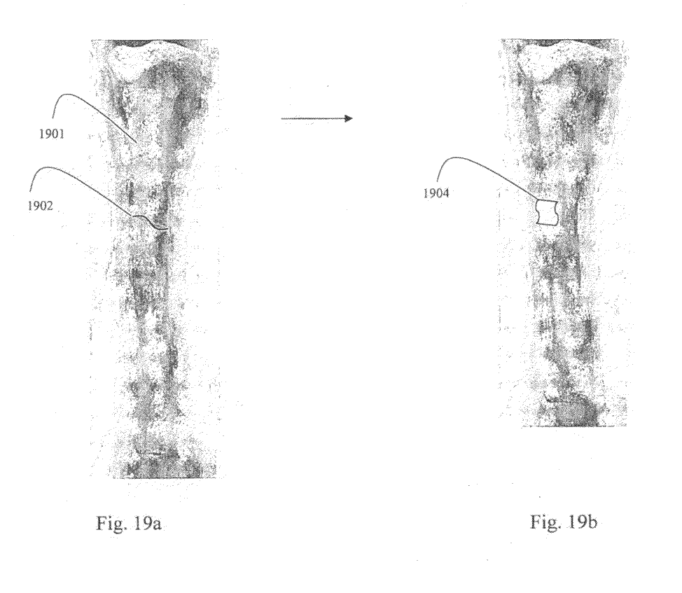

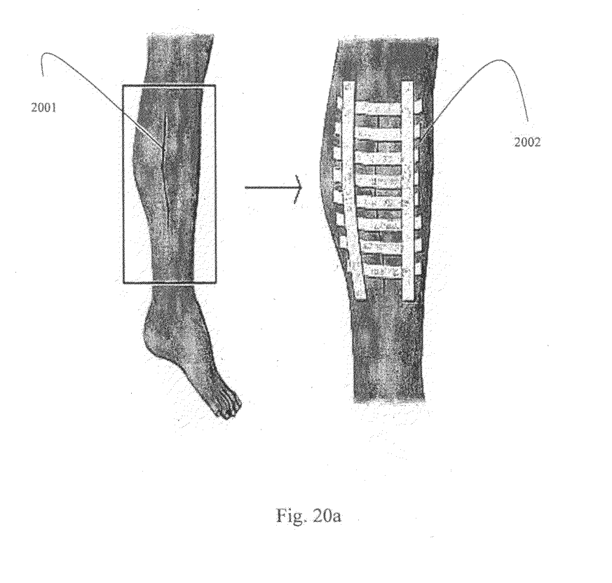

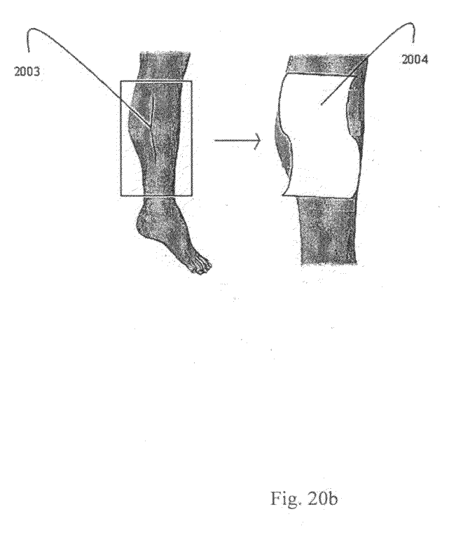

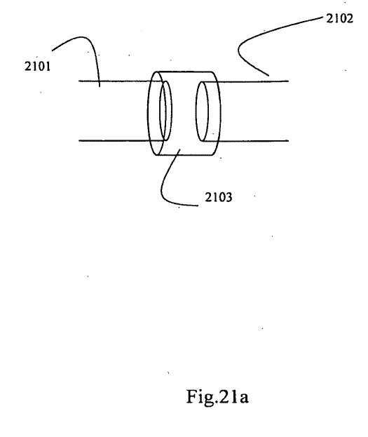

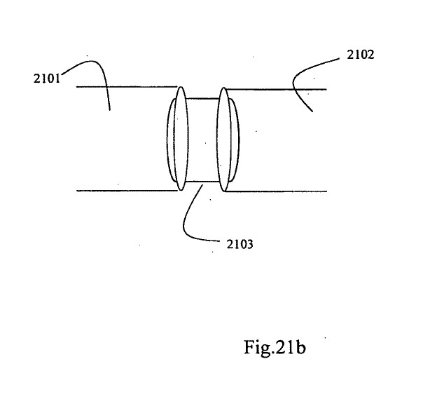

In FIG. 15a,b a similar embodiment is shown wherein anchors 1501, 1502 are forced together or apart by expanding or collapsing the element 1503 by means of internal pressure. In FIG. 15b an embodiment is shown where the element 1503 is not perpendicular to the anchor elements, providing the tilted configuration shown.

The present invention also provides an implantable medical device adapted for use in surgical operations. The device being implantable in a predetermined body portion to be treated, said predetermined body portion is characterized by a first extremity and by a second extremity. The device comprises (a) at least one shaft having a proximal end and a distal end; said shaft is provided with ratchets located on said distal end of said shaft; (b) at least one stationary anchoring means coupled to said shaft in said proximal end, said stationary anchoring means is adapted to be at least partially reversibly anchored to said first extremity in said predetermined body portion; and, (c) at least one movable anchoring means coupled to said distal end of said shaft; said movable anchoring means is adapted to be at least partially reversibly anchored to said second extremity in said predetermined body portion; said movable anchoring means are adapted to unidirectionally move within said shaft such that the distance between said first extremity and said second extremity is alterable.

According to another embodiment the implantable medical device additionally comprising means coupled to said movable anchoring means and enables said movement of said movable anchoring means along the longitudinal axis of said shaft.

According to another embodiment the surgical operations are selected from a group consisting of cosmetic surgeries, especially for remoulding and/or reconstructing both soft and/or hard tissues, repairing apertures in soft biological tissue or any combination thereof. According to another embodiment the implantable medical device is useful in cosmetic medical procedure selected from a group consisting of wrinkles removal, face lifting, in medical procedure selected from a group consisting of intensive and/or immediate care for repairing aperture, in medical procedure for repair aperture in hard biological tissue selected from a group consisting of orthopaedic surgeries, dental surgeries, soft and hard tissues reattachments.

According to another embodiment the at least two anchoring means are positioned at an angle A relatively to each other. Angle A is greater than about 0 degrees and lower than about 180 degrees, especially at an angle of 0 degrees.

FIG. 16 illustrates the above mentioned embodiment. In FIG. 16 anchors 1601 (i.e., stationary anchoring means), 1604 (i.e., movable anchoring means) are forced together or apart by rotating the rigid rod 1602 (i.e., the shaft), which is provided with screw 1603 (i.e., ratchets) that fits a complementary thread on anchor 1604. Thus when the rigid rod 1602 is rotated relative to anchor 1604 the anchor can be pulled or pushed along the length of the rod, providing an exact amount of tension desired. FIG. 16b gives a close up of the mechanism. In the alternative embodiment shown in FIG. 16c, the movement is provided by means of piston 1605 and ratchets 1606. It is optionally that once the movable anchoring means (i.e., 1604) are positioned at the desirable location, the piston 1605 is removed Out of the body.

In FIG. 17a an embodiment is shown wherein a sliding element 1701 is adapted to slide along arm 1702. This forces connecting member 1705 to force arm 1703 away from arm 1702, thus opening the arms and increasing the angle between them. Cog elements 1704 provide for anchoring the arms to tissue. FIG. 17b illustrates the same, except in the ‘closed’ disposition.

In the alternative embodiment of FIG. 17c the actuating element 1706 is an inflatable member.

FIG. 17d illustrates the same, except in the ‘closed’ disposition.

With reference to FIG. 18 one embodiment of the device is illustrated. The implantable medical device anchor elements 1801 are placed subcutaneously using the introducing device. Sutures 1802 connect each anchor element to its next neighbor, and can be tightened to the desired tension, independent of the tension in the other segments of the suture. The sutures 1802 are kept under tension while the sutures 1803 are relaxed allowing for a directional and cooperative effect to be achieved using several anchoring devices.

The following description is provided in order to demonstrate further uses of the present invention.

The first example applies to hard tissues (e.g. bones). The method allows for reconstruction and/or fixed relative placement of broken or fractured bones to allow for accelerated healing.

In FIG. 19 a possible use of the invention is shown. A break 1902 in a bone 1901 is repaired by use of patch 1904 containing a set of the implantable medical devices as any of the above mentioned embodiments tending to draw the bone parts together to facilitate healing.

The method consists of placing the patch 1904 into place, and affixing the traction elements of one side into place on the bone. Once these traction elements are anchored, the traction elements on the other side of the break are anchored. Then the tensioning elements are activated, e.g. by drawing closed the suture threaded through the plurality of implantable medical device traction elements (for the embodiment using implantable medical devices with threaded sutures).

The second example is the use of the invention from repairing biological apertures.

In FIG. 20 a possible embodiment of this example is shown. A cut 2001 in the skin is repaired by use of a use of patch 2002 containing a set of the implantable medical devices as any of the above mentioned embodiments tending to draw the skin parts together to facilitate healing.

The method comprises using a patch containing the above mentioned implantable medical devices or alternative traction and tensioning members, embedded within said patch. The patch is place upon the aperture, the implantable medical devices are anchored, and the sutures or other traction devices are activated and thus the aperture is closed.

In FIG. 20b another possible use of the invention is shown. A cut 2003 in the skin is repaired by use of a patch 2004 containing a set of the implantable medical devices as any of the above mentioned embodiments tending to draw the skin parts together to facilitate healing.

The method comprises using a sheet containing the above mentioned implantable medical devices or alternative traction and tensioning members, embedded within said patch. The patch is place upon the aperture, the implantable medical devices are anchored, and the sutures or other traction devices are activated and thus the aperture is closed.

The third example is the use of the invention in the field of anastomosis.

FIG. 21 illustrates another possible use of the invention. Two blood vessel halves 2101, 2102 of a ruptured blood vessel are brought into fluid communication by exterior connecting element 2103 which comprises the implantable medical devices as any of the above mentioned embodiments.

The method comprises placing a patch around the outer surface of the vessels. This patch contains the above mentioned implantable medical devices or alternative traction and tensioning members, embedded within said patch. The patch is place around the anastomosis, the implantable medical devices are anchored, and the sutures or other traction devices are activated and thus the anastomosis is closed.

In FIG. 21b a possible use of the invention is shown. Two blood vessel halves 2101, 2102 of a ruptured blood vessel are brought into fluid communication by interior connecting element 2103 which comprises the implantable medical devices as any of the above mentioned embodiments.

The method comprises placing a patch within the inner surface of the vessels. This patch contains the above mentioned implantable medical devices or alternative traction and tensioning members, embedded within said patch. The patch is place around the anastomosis, the implantable medical devices are anchored, and the sutures or other traction devices are activated and thus the anastomosis is closed. This embodiment is especially good for preventing collapse of the vessel.

It is within provision of the invention that the means for affixing the various elements of the device within the body may be accomplished by a number of means. For example, elements may be affixed remotely by means of an introducer, or in situ by hand. The particular method of affixing may be selected from a group tying, connecting, attaching, gluing, stapling, and sewing.

The devices can be applied to the skin, subcutaneous tissues and all deep tissues as well. In addition, they can be applied to normal tissues in their natural, in situ form, or to disrupted tissues, such a wounds caused by any trauma or surgical cut in, for example, the head, facial and neck skin, chest, abdomen, torso, and upper and lower limbs. It can be applied to the abdominal organs, lungs and other organs of the body. The devices can be used for tissue and organ approximation.

1-86. (canceled)

87. An implantable medical device adapted for use in surgical operations, said device being implantable in a predetermined body portion to be treated; said predetermined body portion is characterized by a first extremity and by a second extremity, wherein said device comprises:
at least one spring-like element having a body, a distal end, and a proximal end; said body being characterized by (i) at least one inactivated position; and, (ii) a plurality of activated positions;
at least one anchoring means coupled to said distal end of said element, adapted to anchor said distal end to said first extremity in said predetermined body portion; and,
at least one anchoring means coupled to said proximal end of said element, adapted to anchor said proximal end to said second extremity in said predetermined body portion;
said element is at least partially reconfigurable from said activated position to said inactivated position and/or from said plurality of inactivated positions to said plurality of activated positions such that the distance between said first extremity and said second extremity is either increased or decreased.

88. The implantable medical device according to claim 87, wherein said surgical operations are selected from a group consisting of intensive and/or immediate care for repairing apertures, repairing apertures in hard biological tissue selected from a group consisting of orthopaedic surgeries, dental surgeries, soft and hard tissues reattachments, cosmetic surgeries, especially for wrinkles removal, face lifting, remoulding and/or reconstructing both soft and/or hard tissues, repairing apertures in soft biological tissue or any combination thereof.

89. The implantable medical device according to claim 87, wherein said at least two anchoring means are positioned at an angle A relatively to each other; said angle A is greater than about 0 degrees and lower than about 180 degrees, especially at an angle 180 degrees.

90. The implantable medical device according to claim 87, wherein said spring-like element is made of material selected from a group consisting of shape memory materials, biodegradable materials, biocompatible materials or any combination thereof.

91. An implantable medical device adapted for use in surgical operations; said device being implantable in a predetermined body portion to be treated; said predetermined body portion is characterized by a first extremity and by a second extremity; wherein said device comprises:
at least one stationary anchoring means adapted to be at least partially reversibly anchored to said first extremity in said predetermined body portion;
at least one movable anchoring means in mechanical communication with said stationary anchoring means via at least one shaft; said movable anchoring means is adapted to be at least partially reversibly anchored to said second extremity in said predetermined body portion;
said movable anchoring means is adapted to reciprocally move in a continues manner along the longitudinal axis of said shaft such that the distance between said first extremity and said second extremity is alterable; said stationary anchoring means and said movable anchoring means are positioned at an angle A relatively to each other;
said angle A is greater than about 0 degrees and lower than about 180 degrees, especially at an angle of 0 degrees;
at least one inflatable or fillable element having a body, a distal end and a proximal end; said body is characterized by at least one inactivated position and a plurality of activated positions; at least one end of said element is coupled to said movable anchoring means; said element is at least partially reconfigurable from said activated position to said inactivated position and/or from said plurality of inactivated positions to said plurality of activated positions; and,
activating means selected from a group consisting of inflating or deflating means, filling or withdrawing means, pressure, tension, force, heat, luminescence, change of Ph, application of magnetic field, application of electric field, voltage adapted to for activate said element such that said body is reconfigured from said activated position to said inactivated position and/or from said inactivated position to said activated position.

92. The implantable medical device according to claim 91, wherein said surgical operations are selected from a group consisting of cosmetic surgeries selected from a group consisting of wrinkles removal, face lifting, especially for remoulding and/or reconstructing both soft and/or hard tissues, repairing apertures in soft biological tissue or any combination thereof.

93. The implantable medical device according to claim 91, useful in medical procedure selected from a group consisting of intensive and/or immediate care for repairing apertures, repairing aperture in hard biological tissue selected from a group consisting of orthopaedic surgeries, dental surgeries, soft and hard tissues reattachments.

94. The implantable medical device according to claim 91, wherein said element is made of material selected from a group consisting of shape memory materials, biodegradable materials, biocompatible materials or any combination thereof.

95. A method for treating tissues in a predetermined body portion during surgical operations, said method comprising steps of:
providing an implantable medical device comprising (i) at least one spring like element having a body, a distal end, and a proximal end; said body is characterized by (i) at least one inactivated position; and, (ii) a plurality of activated positions; (ii) at least one anchoring means coupled to said distal end of said element; and, (iii) at least one anchoring means coupled to said proximal end of said element;
introducing said implantable medical device in said activated position or in said uncativated position to said body portion to be treated;
at least partially reversibly anchoring said distal end of said implantable medical device to said first extremity in said predetermined body portion via one of said anchoring means;
at least partially reversibly anchoring said proximal end of said implantable medical device to said second extremity in said predetermined body portion via one of said anchoring means;
reconfiguring said body from said activated position to said inactivated position and/or from said inactivated position to said activated position;
imparting tension and altering the distance between said first extremity and said second extremity by increasing or decreasing said distance thereby treating said tissue of said body portion.

96. A method for treating tissues in a predetermined body portion during surgical operations, said predetermined body portion to be treated is characterized by a first extremity and a second extremity, said method comprising steps of:
providing an implantable medical device having (i) at least one stationary anchoring means; (ii) at least one movable anchoring means in mechanical communication with said stationary anchoring means via at least one shaft; (iii) at least one inflatable or fillable element; (iv) activating means;
coupling at least one end of said element to said movable anchoring means; said element is at least partially reconfigurable from said activated position to said inactivated position and/or from said plurality of inactivated positions to said plurality of activated positions;
inserting said implantable medical device into said body portion to be treated;
at least partially reversibly anchoring said stationary anchoring means to said first extremity in said body portion to be treated;
at least partially reversibly anchoring said movable anchoring means to said second extremity in said body portion to be treated; and,
activating said element via said activating means;
reciprocally moving said movable anchoring means along said shaft;
thereby imparting tension and altering the distance between said first extremity and said second extremity thus, treating said tissue of said body portion.

97. An implantable medical device adapted for use in surgical operations; said device being implantable in a predetermined body portion to be treated; wherein said device comprises:
at least one hollow element selected from a spring, an inflatable element, fillable element or any combination thereof; said element having a biocompatible outer surface, said hollow element is characterised by an inactivated position and a plurality of activated positions;
a plurality of protrusions coupled to said outer surface, adapted to anchor said hollow element to said body portion to be treated;
coupling means accommodated within said hollow element, for imparting mechanical tension on said element.

98. The implantable medical device according to claim 97, wherein the shape of said element is selected from a group consisting of: zigzag shaped, square shape, C shape, of regular or irregular spiral, lumen, pipe, crescent, linear, bagel-like (annular), or star-like cross section, planar or proximally planar shaped barriers of any size, shape or type, 3D configurations, and multi-dimensional configurations comprising a plurality of regular or irregular two or more 2D planes and/or 3D spatial members interlinked together to form a continuous member.

99. The implantable medical device according to claim 97, wherein said surgical operations are selected from a group consisting of cosmetic surgeries selected from a group consisting of wrinkles removal, face lifting, remoulding and/or reconstructing both soft and/or hard tissues, repairing apertures in soft biological tissue, intensive and/or immediate care for repairing apertures in hard biological tissue selected from a group consisting of orthopaedic surgeries, dental surgeries, soft and hard tissues reattachments or any combination thereof.

100. The implantable medical device according to claim 97, wherein said elements are made of material selected from a group consisting of shape memory materials, biodegradable materials, biocompatible materials or any combination thereof.

101. A method for treating tissues in a predetermined body portion during surgical operations, wherein said method comprising steps of:
providing at least two implantable medical device, each of which comprises (i) a hollow element having a biocompatible outer surface, said hollow element is characterised by an inactivated position and a plurality of activated positions; (ii) a plurality of protrusions coupled to said outer surface, adapted to anchor said hollow element to said body portion to be treated; and, (iii) coupling means accommodated within said hollow element, for imparting mechanical tension on said element;
threading said coupling means through said hollow element thereby mechanically coupling pairs of said implantable medical device together;
incorporating said threaded implantable medical devices in said inactivated position within an introducer;
introducing at least one of said implantable medical device via said introducer into said body portion to be treated;
at least partially anchoring said implantable medical device to said tissue in said body portion to be treated via said plurality of protrusions;
introducing the second element via said introducer into said body portion to be treated;
at least partially anchoring said second element to said tissue in said body portion to be treated via said plurality of cogs;
altering the tension in said coupling means such that the distance between said pairs is altered and mechanical tension is imparted on said tissue, thereby treating said tissue in said predetermined body portion.

102. The method according to claim 101, wherein said step of tensing said tissue is performed by altering in an arbitrary geometric manner said pairs by means of independently fixed tension between every connected pair of said elements.

103. The method according to claim 101, additionally comprising step of affixing each of said flexible coupling means to said tissues in said predetermined body portion.

104. A device for treating tissues in a predetermined body portion, said device comprising:
a magazine accommodating a plurality of implantable medical devices threaded together via coupling means;
means for individually introducing each one of said implantable medical devices to said predetermined body portion;
means for imparting mechanical tension in said coupling means between each pair of implantable medical device by stretching or relaxing said coupling means.

105. The device of claim 104, wherein said means for individually introducing comprises (a) a shaft adapted to linearly move said implantable medical devices; and, (b) a stopper adapted to introduce a single implantable medical device to said body portion to be treated.

106. A fastener for use in surgical operations; said fastener being implantable in a predetermined body portion to be treated, wherein said fastener comprises:
at least two arm positioned at an angle A relatively to one another;
said angle A is greater than about 0 and lower than about 180 degrees;
a rigid stopper adapted to fixate said at least two arms at said angle A;
a plurality of protrusions coupled to at least a portion of said arms, adapted to anchor said fastener to said tissue in said body portion to be treated.

107. The fastener according to claim 106, wherein said surgical operations are selected from a group consisting of cosmetic surgeries, wrinkles removal, face lifting, remoulding and/or reconstructing both soft and/or hard tissues, repairing apertures in soft biological tissue, orthopaedic surgeries, dental surgeries, soft and hard tissues reattachments or any combination thereof.
